SEQUENCE LISTING (76 sequences)

SEQ ID NO: 1 - protein, 698 aa, Artificial
  PCSK9 with his tag PCSK9-His6, used as an immunogen for immunizing mice or used as a detection reagent
MGTVSSRRSWWPLPLLLLLLLLLGPAGARAQEDEDGDYEELVLALRSEEDGLAEAPEHGTTATFHRCAKDPWRLPGTYVVVLKEETHLSQSERTARRLQAQAARRGYLTKILHVFHGLLPGFLVKMSGDLLELALKLPHVDYIEEDSSVFAQSIPWNLERITPPRYRADEYQPPDGGSLVEVYLLDTSIQSDHREIEGRVMVTDFENVPEEDGTRFHRQASKCDSHGTHLAGVVSGRDAGVAKGASMRSLRVLNCQGKGTVSGTLIGLEFIRKSQLVQPVGPLVVLLPLAGGYSRVLNAACQRLARAGVVLVTAAGNFRDDACLYSPASAPEVITVGATNAQDQPVTLGTLGTNFGRCVDLFAPGEDIIGASSDCSTCFVSQSGTSQAAAHVAGIAAMMLSAEPELTLAELRQRLIHFSAKDVINEAWFPEDQRVLTPNLVAALPPSTHGAGWQLFCRTVWSAHSGPTRMATAVARCAPDEELLSCSSFSRSGKRRGERMEAQGGKLVCRAHNAFGGEGVYAIARCCLLPQANCSVHTAPPAEASMGTRVHCHQQGHVLTGCSSHWEVEDLGTHKPPVLRPRGQPNQCVGHREASIHASCCHAPGLECKVKEHGIPAPQEQVTVACEEGWTLTGCSALPGTSHVLGAYAVDNTCVVRSRDVSTTGSTSEGAVTAVAICCRSRHLAQASQELQHHHHHH

SEQ ID NO: 2 - protein, 714 aa, ARTIFICIAL
  PCSK9 with PADRE peptide and his tag PCSK9- PADRE-His6, used as an immunogen wherein the contained PADRE peptide can promote immunization
MGTVSSRRSWWPLPLLLLLLLLLGPAGARAQEDEDGDYEELVLALRSEEDGLAEAPEHGTTATFHRCAKDPWRLPGTYVVVLKEETHLSQSERTARRLQAQAARRGYLTKILHVFHGLLPGFLVKMSGDLLELALKLPHVDYIEEDSSVFAQSIPWNLERITPPRYRADEYQPPDGGSLVEVYLLDTSIQSDHREIEGRVMVTDFENVPEEDGTRFHRQASKCDSHGTHLAGVVSGRDAGVAKGASMRSLRVLNCQGKGTVSGTLIGLEFIRKSQLVQPVGPLVVLLPLAGGYSRVLNAACQRLARAGVVLVTAAGNFRDDACLYSPASAPEVITVGATNAQDQPVTLGTLGTNFGRCVDLFAPGEDIIGASSDCSTCFVSQSGTSQAAAHVAGIAAMMLSAEPELTLAELRQRLIHFSAKDVINEAWFPEDQRVLTPNLVAALPPSTHGAGWQLFCRTVWSAHSGPTRMATAVARCAPDEELLSCSSFSRSGKRRGERMEAQGGKLVCRAHNAFGGEGVYAIARCCLLPQANCSVHTAPPAEASMGTRVHCHQQGHVLTGCSSHWEVEDLGTHKPPVLRPRGQPNQCVGHREASIHASCCHAPGLECKVKEHGIPAPQEQVTVACEEGWTLTGCSALPGTSHVLGAYAVDNTCVVRSRDVSTTGSTSEGAVTAVAICCRSRHLAQASQELQGSGAKFVAAWTLKAAAHHHHHH

SEQ ID NO: 3 - protein, 704 aa, ARTIFICIAL
  Fusion protein of PCSK9 containing TEV cleavage site and his tag PCSK9-TEV-His6, as an immunogen, N-PCSK9 (N-terminal PCSK9 domain) can be obtained via TEV enzyme digestion
MGTVSSRRSWWPLPLLLLLLLLLGPAGARAQEDEDGDYEELVLALRSEEDGLAEAPEHGTTATFHRCAKDPWRLPGTYVVVLKEETHLSQSERTARRLQAQAARRGYLTKILHVFHGLLPGFLVKMSGDLLELALKLPHVDYIEEDSSVFAQSIPWNLERITPPRYRADEYQPPDGGSLVEVYLLDTSIQSDHREIEGRVMVTDFENVPEEDGTRFHRQASKCDSHGTHLAGVVSGRDAGVAKGASMRSLRVLNCQGKGTVSGTLIGLEFIRKSQLVQPVGPLVVLLPLAGGYSRVLNAACQRLARAGVVLVTAAGNFRDDACLYSPASAPEVITVGATNAQDQPVTLGTLGTNFGRCVDLFAPGEDIIGASSDCSTCFVSQSGTSQAAAHVAGIAAMMLSAEPELTLAELRQRLIHFSAKDVINEAWFPEDQRVLTPNLVAALPPSTHENLYFQGAGWQLFCRTVWSAHSGPTRMATAVARCAPDEELLSCSSFSRSGKRRGERMEAQGGKLVCRAHNAFGGEGVYAIARCCLLPQANCSVHTAPPAEASMGTRVHCHQQGHVLTGCSSHWEVEDLGTHKPPVLRPRGQPNQCVGHREASIHASCCHAPGLECKVKEHGIPAPQEQVTVACEEGWTLTGCSALPGTSHVLGAYAVDNTCVVRSRDVSTTGSTSEGAVTAVAICCRSRHLAQASQELQHHHHHH

SEQ ID NO: 4 - protein, 698 aa, ARTIFICIAL
  PCSK9-D374Y mutant protein with his tag PCSK9-D374Y-His6, as a detection reagent
MGTVSSRRSWWPLPLLLLLLLLLGPAGARAQEDEDGDYEELVLALRSEEDGLAEAPEHGTTATFHRCAKDPWRLPGTYVVVLKEETHLSQSERTARRLQAQAARRGYLTKILHVFHGLLPGFLVKMSGDLLELALKLPHVDYIEEDSSVFAQSIPWNLERITPPRYRADEYQPPDGGSLVEVYLLDTSIQSDHREIEGRVMVTDFENVPEEDGTRFHRQASKCDSHGTHLAGVVSGRDAGVAKGASMRSLRVLNCQGKGTVSGTLIGLEFIRKSQLVQPVGPLVVLLPLAGGYSRVLNAACQRLARAGVVLVTAAGNFRDDACLYSPASAPEVITVGATNAQDQPVTLGTLGTNFGRCVDLFAPGEDIIGASSYCSTCFVSQSGTSQAAAHVAGIAAMMLSAEPELTLAELRQRLIHFSAKDVINEAWFPEDQRVLTPNLVAALPPSTHGAGWQLFCRTVWSAHSGPTRMATAVARCAPDEELLSCSSFSRSGKRRGERMEAQGGKLVCRAHNAFGGEGVYAIARCCLLPQANCSVHTAPPAEASMGTRVHCHQQGHVLTGCSSHWEVEDLGTHKPPVLRPRGQPNQCVGHREASIHASCCHAPGLECKVKEHGIPAPQEQVTVACEEGWTLTGCSALPGTSHVLGAYAVDNTCVVRSRDVSTTGSTSEGAVTAVAICCRSRHLAQASQELQHHHHHH

SEQ ID NO: 5 - protein, 719 aa, ARTIFICIAL
  PCSK9 protein inserted with biotin receiving peptide and his tag PCSK9-BP15-His6. As a detection reagent, biotin will be labeled to the position of BP15 peptide during the expression, avoiding the biotin labeling in vitro and consequently avoiding possible conformational changes
MGTVSSRRSWWPLPLLLLLLLLLGPAGARAQEDEDGDYEELVLALRSEEDGLAEAPEHGTTATFHRCAKDPWRLPGTYVVVLKEETHLSQSERTARRLQAQAARRGYLTKILHVFHGLLPGFLVKMSGDLLELALKLPHVDYIEEDSSVFAQSIPWNLERITPPRYRADEYQPPDGGSLVEVYLLDTSIQSDHREIEGRVMVTDFENVPEEDGTRFHRQASKCDSHGTHLAGVVSGRDAGVAKGASMRSLRVLNCQGKGTVSGTLIGLEFIRKSQLVQPVGPLVVLLPLAGGYSRVLNAACQRLARAGVVLVTAAGNFRDDACLYSPASAPEVITVGATNAQDQPVTLGTLGTNFGRCVDLFAPGEDIIGASSDCSTCFVSQSGTSQAAAHVAGIAAMMLSAEPELTLAELRQRLIHFSAKDVINEAWFPEDQRVLTPNLVAALPPSTHGAGWQLFCRTVWSAHSGPTRMATAVARCAPDEELLSCSSFSRSGKRRGERMEAQGGKLVCRAHNAFGGEGVYAIARCCLLPQANCSVHTAPPAEASMGTRVHCHQQGHVLTGCSSHWEVEDLGTHKPPVLRPRGQPNQCVGHREASIHASCCHAPGLECKVKEHGIPAPQEQVTVACEEGWTLTGCSALPGTSHVLGAYAVDNTCVVRSRDVSTTGSTSEGAVTAVAICCRSRHLAQASQELQGSTSGSGLNDIFEAQKIEWHEHHHHHH

SEQ ID NO: 6 - protein, 719 aa, ARTIFICIAL
  PCSK9 D374Y mutant protein inserted with biotin receiving peptide and his tag PCSK9-D374Y-BP15-His6, as a detection protein
MGTVSSRRSWWPLPLLLLLLLLLGPAGARAQEDEDGDYEELVLALRSEEDGLAEAPEHGTTATFHRCAKDPWRLPGTYVVVLKEETHLSQSERTARRLQAQAARRGYLTKILHVFHGLLPGFLVKMSGDLLELALKLPHVDYIEEDSSVFAQSIPWNLERITPPRYRADEYQPPDGGSLVEVYLLDTSIQSDHREIEGRVMVTDFENVPEEDGTRFHRQASKCDSHGTHLAGVVSGRDAGVAKGASMRSLRVLNCQGKGTVSGTLIGLEFIRKSQLVQPVGPLVVLLPLAGGYSRVLNAACQRLARAGVVLVTAAGNFRDDACLYSPASAPEVITVGATNAQDQPVTLGTLGTNFGRCVDLFAPGEDIIGASSYCSTCFVSQSGTSQAAAHVAGIAAMMLSAEPELTLAELRQRLIHFSAKDVINEAWFPEDQRVLTPNLVAALPPSTHGAGWQLFCRTVWSAHSGPTRMATAVARCAPDEELLSCSSFSRSGKRRGERMEAQGGKLVCRAHNAFGGEGVYAIARCCLLPQANCSVHTAPPAEASMGTRVHCHQQGHVLTGCSSHWEVEDLGTHKPPVLRPRGQPNQCVGHREASIHASCCHAPGLECKVKEHGIPAPQEQVTVACEEGWTLTGCSALPGTSHVLGAYAVDNTCVVRSRDVSTTGSTSEGAVTAVAICCRSRHLAQASQELQGSTSGSGLNDIFEAQKIEWHEHHHHHH

SEQ ID NO: 7 - protein, 802 aa, ARTIFICIAL
  PCSK9 receptor protein LDLR extracellular domain with Flag tag and his tag LDLR-ECD-Flag-His6 as a detection reagent
MGPWGWKLRWTVALLLAAAGTAVGDRCERNEFQCQDGKCISYKWVCDGSAECQDGSDESQETCLSVTCKSGDFSCGGRVNRCIPQFWRCDGQVDCDNGSDEQGCPPKTCSQDEFRCHDGKCISRQFVCDSDRDCLDGSDEASCPVLTCGPASFQCNSSTCIPQLWACDNDPDCEDGSDEWPQRCRGLYVFQGDSSPCSAFEFHCLSGECIHSSWRCDGGPDCKDKSDEENCAVATCRPDEFQCSDGNCIHGSRQCDREYDCKDMSDEVGCVNVTLCEGPNKFKCHSGECITLDKVCNMARDCRDWSDEPIKECGTNECLDNNGGCSHVCNDLKIGYECLCPDGFQLVAQRRCEDIDECQDPDTCSQLCVNLEGGYKCQCEEGFQLDPHTKACKAVGSIAYLFFTNRHEVRKMTLDRSEYTSLIPNLRNVVALDTEVASNRIYWSDLSQRMICSTQLDRAHGVSSYDTVISRDIQAPDGLAVDWIHSNIYWTDSVLGTVSVADTKGVKRKTLFRENGSKPRAIVVDPVHGFMYWTDWGTPAKIKKGGLNGVDIYSLVTENIQWPNGITLDLLSGRLYWVDSKLHSISSIDVNGGNRKTILEDEKRLAHPFSLAVFEDKVFWTDIINEAIFSANRLTGSDVNLLAENLLSPEDMVLFHNLTQPRGVNWCERTTLSNGGCQYLCLPAPQINPHSPKFTCACPDGMLLARDMRSCLTEAEAAVATQETSTVRLKVSSTAVRTQHTTTRPVPDTSRLPGATPGLTTVEIVTMSHQALGDVAGRGNEKKPSSVRDYKDDDDKHHHHHH

SEQ ID NO: 8 - protein, 331 aa, ARTIFICIAL
  Fusion protein of truncated LDLR extracellular domain and hIgG1 Fc (with PCSK9 binding activity) LDLR-sECD-Fc (hIgG1) as a detection reagent
MEFGLSWLFLVAILKGVQCGTNECLDNNGGCSHVCNDLKIGYECLCPDGFQLVAQRRCEDIDECQDPDTCSQLCVNLEGGYKCQCEEGFQLDPHTKACKEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 294 aa, ARTIFICIAL
  Fusion protein of further truncated LDLR extracellular domain and hIgG1 Fc (with PCSK9 binding activity) LDLR-ssECD-Fc (hIgG1) as a detection reagent
MEFGLSWLFLVAILKGVQCGTNECLDNNGGCSHVCNDLKIGYECLCPDGFQLVAQRRCEDIDEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 121 aa, ARTIFICIAL
  h011-1 VH
QVQLVQSGAEVKKPGASVKVSCKASGYTFTGYTIHWVRQAPGQGLEWMGYINPSSTYTKFNQKFKDRVTMTRDTSISTAYMELSRLRSDDTAVYYCARERIYSNYWFFDVWGQGTTVTVSS

SEQ ID NO: 11 - protein, 107 aa, ARTIFICIAL
  h011-1 VL
DIQMTQSPSSLSASVGDRVTITCKASQNVYTAVAWYQQKPGKAPKLLIYSASNRYTGVPSRFSGSGSGTDFTLTISSLQPEDFATYYCQQYSSYPYTFGGGTKVEIK

SEQ ID NO: 12 - protein, 121 aa, ARTIFICIAL; identical to SEQ ID NO: 10
  h011-3 VH

SEQ ID NO: 13 - protein, 107 aa, ARTIFICIAL
  h011-3 VL
DIVMTQSPSSLSASVGDRVTITCKASQNVYTAVAWYQQKPGKSPKLLIYSASNRYTGVPSRFSGSGSGTDFTLTISSLQPEDFATYFCQQYSSYPYTFGGGTKVEIK

SEQ ID NO: 14 - protein, 5 aa, Murine
GYTIH

SEQ ID NO: 15 - protein, 17 aa, Murine
YINPSSTYTKFNQKFKD

SEQ ID NO: 16 - protein, 14 aa, Murine
ARERIYSNYWFFDV

SEQ ID NO: 17 - protein, 11 aa, Murine
KASQNVYTAVA

SEQ ID NO: 18 - protein, 7 aa, Murine
SASNRYT

SEQ ID NO: 19 - protein, 9 aa, Murine
QQYSSYPYT

SEQ ID NO: 20 - protein, 5 aa, Artificial
  HCDR1-1
GYDIH

SEQ ID NO: 21 - protein, 5 aa, Artificial
  HCDR1-2
GYEIH

SEQ ID NO: 22 - protein, 17 aa, Artificial
  HCDR2-1
EILPSSTYTKFNQKFKD

SEQ ID NO: 23 - protein, 17 aa, Artificial
  HCDR2-2
EINPSGTYTKFNQKFKD

SEQ ID NO: 24 - protein, 17 aa, Artificial
  HCDR2-3
YINPSATYTKFNQKFKD

SEQ ID NO: 25 - protein, 17 aa, Artificial
  HCDR2-4
EIIPSSTYTKFNQKFKD

SEQ ID NO: 26 - protein, 17 aa, Artificial
  HCDR2-5
EINPSSTYTKFNQKFKD

SEQ ID NO: 27 - protein, 17 aa, Artificial
  HCDR2-6
YIVPSSTYTKFNQKFKD

SEQ ID NO: 28 - protein, 14 aa, Artificial
  HCDR3-1
ARENIYFNYWFFDR

SEQ ID NO: 29 - protein, 14 aa, Artificial
  HCDR3-2
ARENIFSNYWFFDR

SEQ ID NO: 30 - protein, 14 aa, Artificial
  HCDR3-3
ARERIFSNYWFFDV

SEQ ID NO: 31 - protein, 11 aa, Artificial
  LCDR1-1
KASQNVYWEVD

SEQ ID NO: 32 - protein, 11 aa, Artificial
  LCDR1-2
KASQNVYWEVV

SEQ ID NO: 33 - protein, 11 aa, Artificial
  LCDR1-3
KASQNVYTEVA

SEQ ID NO: 34 - protein, 11 aa, Artificial
  LCDR1-4
KASQNVYTAVD

SEQ ID NO: 35 - protein, 7 aa, Artificial
  LCDR2-1
EMVNRYT

SEQ ID NO: 36 - protein, 7 aa, Artificial
  LCDR2-2
EASNRYT

SEQ ID NO: 37 - protein, 7 aa, Artificial
  LCDR2-3
QASNRYT

SEQ ID NO: 38 - protein, 9 aa, Artificial
  LCDR3-1
QQFSWFPYT

SEQ ID NO: 39 - protein, 9 aa, Artificial
  LCDR3-2
QQLSSQPET

SEQ ID NO: 40 - protein, 9 aa, Artificial
  LCDR3-3
QQLSSYPDT

SEQ ID NO: 41 - protein, 9 aa, Artificial
  LCDR3-4
QQLSSSPET

SEQ ID NO: 42 - protein, 9 aa, Artificial
  LCDR3-5
QQLSSYPYT

SEQ ID NO: 43 - protein, 5 aa, Artificial
  Synthetic peptide
  misc_feature (3)..(3): Xaa is Thr, Asp or Glu
GYXIH

SEQ ID NO: 44 - protein, 17 aa, Artificial
  Synthetic peptide
  misc_feature (1)..(1): Xaa is Tyr or Glu
  misc_feature (3)..(3): Xaa is Asn,Leu,Ile or Val
  misc_feature (6)..(6): Xaa is Ser,Gly or Ala
XIXPSXTYTKFNQKFKD

SEQ ID NO: 45 - protein, 14 aa, Artificial
  Synthetic peptide
  misc_feature (4)..(4): Xaa is Arg or Asn
  misc_feature (6)..(6): Xaa is Tyr or Phe
  misc_feature (7)..(7): Xaa is Ser or Phe
  misc_feature (14)..(14): Xaa is Val or Arg
AREXIXXNYWFFDX

SEQ ID NO: 46 - protein, 11 aa, Artificial
  Synthetic peptide
  misc_feature (8)..(8): Xaa is Thr or Trp
  misc_feature (9)..(9): Xaa is Ala or Glu
  misc_feature (11)..(11): Xaa is Ala,Asp or Val
KASQNVYXXVX

SEQ ID NO: 47 - protein, 7 aa, Artificial
  Synthetic peptide
  misc_feature (1)..(1): Xaa is Ser, Glu or Gln
  misc_feature (2)..(2): Xaa is Ala or Met
  misc_feature (3)..(3): Xaa is Ser or Val
XXXNRYT

SEQ ID NO: 48 - protein, 9 aa, Artificial
  Synthetic peptide
  misc_feature (3)..(3): Xaa is Tyr,Phe or Leu
  misc_feature (5)..(5): Xaa is Ser or Trp
  misc_feature (6)..(6): Xaa is Tyr,Phe,Gln or Ser
  misc_feature (8)..(8): Xaa is Tyr,Asp or Glu
QQXSXXPXT

SEQ ID NO: 49 - protein, 121 aa, Artificial
  h011-3050-VH
QVQLVQSGAEVKKPGASVKVSCKASGYTFTGYTIHWVRQAPGQGLEWMGEILPSSTYTKFNQKFKDRVTMTRDTSISTAYMELSRLRSDDTAVYYCARERIYSNYWFFDVWGQGTTVTVSS

SEQ ID NO: 50 - protein, 121 aa, Artificial
  h011-3058-VH
QVQLVQSGAEVKKPGASVKVSCKASGYTFTGYTIHWVRQAPGQGLEWMGEINPSGTYTKFNQKFKDRVTMTRDTSISTAYMELSRLRSDDTAVYYCARERIYSNYWFFDVWGQGTTVTVSS

SEQ ID NO: 51 - protein, 121 aa, Artificial
  h011-3065-VH
QVQLVQSGAEVKKPGASVKVSCKASGYTFTGYDIHWVRQAPGQGLEWMGYINPSSTYTKFNQKFKDRVTMTRDTSISTAYMELSRLRSDDTAVYYCARERIYSNYWFFDVWGQGTTVTVSS

SEQ ID NO: 52 - protein, 121 aa, Artificial
  h011-3070-VH
QVQLVQSGAEVKKPGASVKVSCKASGYTFTGYTIHWVRQAPGQGLEWMGYINPSSTYTKFNQKFKDRVTMTRDTSISTAYMELSRLRSDDTAVYYCARENIYFNYWFFDRWGQGTTVTVSS

SEQ ID NO: 53 - protein, 121 aa, Artificial
  h011-3073-VH
QVQLVQSGAEVKKPGASVKVSCKASGYTFTGYTIHWVRQAPGQGLEWMGYINPSSTYTKFNQKFKDRVTMTRDTSISTAYMELSRLRSDDTAVYYCARENIFSNYWFFDRWGQGTTVTVSS

SEQ ID NO: 54 - protein, 121 aa, Artificial
  h011-3133-VH
QVQLVQSGAEVKKPGASVKVSCKASGYTFTGYEIHWVRQAPGQGLEWMGYINPSATYTKFNQKFKDRVTMTRDTSISTAYMELSRLRSDDTAVYYCARERIYSNYWFFDVWGQGTTVTVSS

SEQ ID NO: 55 - protein, 121 aa, Artificial
  h011-3147-VH
QVQLVQSGAEVKKPGASVKVSCKASGYTFTGYTIHWVRQAPGQGLEWMGEIIPSSTYTKFNQKFKDRVTMTRDTSISTAYMELSRLRSDDTAVYYCARERIYSNYWFFDVWGQGTTVTVSS

SEQ ID NO: 56 - protein, 121 aa, Artificial
  h011-3195-VH
QVQLVQSGAEVKKPGASVKVSCKASGYTFTGYTIHWVRQAPGQGLEWMGYINPSSTYTKFNQKFKDRVTMTRDTSISTAYMELSRLRSDDTAVYYCARERIFSNYWFFDVWGQGTTVTVSS

SEQ ID NO: 57 - protein, 121 aa, Artificial
  h011-3190-VH
QVQLVQSGAEVKKPGASVKVSCKASGYTFTGYTIHWVRQAPGQGLEWMGEINPSSTYTKFNQKFKDRVTMTRDTSISTAYMELSRLRSDDTAVYYCARERIYSNYWFFDVWGQGTTVTVSS

SEQ ID NO: 58 - protein, 121 aa, Artificial
  h011-3191-VH
QVQLVQSGAEVKKPGASVKVSCKASGYTFTGYTIHWVRQAPGQGLEWMGYIVPSSTYTKFNQKFKDRVTMTRDTSISTAYMELSRLRSDDTAVYYCARERIYSNYWFFDVWGQGTTVTVSS

SEQ ID NO: 59 - protein, 107 aa, Artificial
  h011-3093-VL
DIVMTQSPSSLSASVGDRVTITCKASQNVYTAVAWYQQKPGKSPKLLIYEMVNRYTGVPSRFSGSGSGTDFTLTISSLQPEDFATYFCQQYSSYPYTFGGGTKVEIK

SEQ ID NO: 60 - protein, 107 aa, Artificial
  h011-3095-VL
DIVMTQSPSSLSASVGDRVTITCKASQNVYTAVAWYQQKPGKSPKLLIYEASNRYTGVPSRFSGSGSGTDFTLTISSLQPEDFATYFCQQYSSYPYTFGGGTKVEIK

SEQ ID NO: 61 - protein, 107 aa, Artificial
  h011-3111-VL
DIVMTQSPSSLSASVGDRVTITCKASQNVYWEVDWYQQKPGKSPKLLIYSASNRYTGVPSRFSGSGSGTDFTLTISSLQPEDFATYFCQQYSSYPYTFGGGTKVEIK

SEQ ID NO: 62 - protein, 107 aa, Artificial
  h011-3118-VL
DIVMTQSPSSLSASVGDRVTITCKASQNVYWEVVWYQQKPGKSPKLLIYSASNRYTGVPSRFSGSGSGTDFTLTISSLQPEDFATYFCQQYSSYPYTFGGGTKVEIK

SEQ ID NO: 63 - protein, 107 aa, Artificial
  h011-3120-VL
DIVMTQSPSSLSASVGDRVTITCKASQNVYTAVAWYQQKPGKSPKLLIYSASNRYTGVPSRFSGSGSGTDFTLTISSLQPEDFATYFCQQFSWFPYTFGGGTKVEIK

SEQ ID NO: 64 - protein, 107 aa, Artificial
  h011-3121-VL
DIVMTQSPSSLSASVGDRVTITCKASQNVYTAVAWYQQKPGKSPKLLIYSASNRYTGVPSRFSGSGSGTDFTLTISSLQPEDFATYFCQQLSSQPETFGGGTKVEIK

SEQ ID NO: 65 - protein, 107 aa, Artificial
  h011-3174-VL
DIVMTQSPSSLSASVGDRVTITCKASQNVYTAVAWYQQKPGKSPKLLIYQASNRYTGVPSRFSGSGSGTDFTLTISSLQPEDFATYFCQQYSSYPYTFGGGTKVEIK

SEQ ID NO: 66 - protein, 107 aa, Artificial
  h011-3181-VL
DIVMTQSPSSLSASVGDRVTITCKASQNVYTAVAWYQQKPGKSPKLLIYSASNRYTGVPSRFSGSGSGTDFTLTISSLQPEDFATYFCQQLSSYPDTFGGGTKVEIK

SEQ ID NO: 67 - protein, 107 aa, Artificial
  h011-3187-VL
DIVMTQSPSSLSASVGDRVTITCKASQNVYTAVAWYQQKPGKSPKLLIYSASNRYTGVPSRFSGSGSGTDFTLTISSLQPEDFATYFCQQLSSSPETFGGGTKVEIK

SEQ ID NO: 68 - protein, 107 aa, Artificial
  h011-3192-VL
DIVMTQSPSSLSASVGDRVTITCKASQNVYTEVAWYQQKPGKSPKLLIYSASNRYTGVPSRFSGSGSGTDFTLTISSLQPEDFATYFCQQYSSYPYTFGGGTKVEIK

SEQ ID NO: 69 - protein, 107 aa, Artificial
  h011-3193-VL
DIVMTQSPSSLSASVGDRVTITCKASQNVYTAVDWYQQKPGKSPKLLIYSASNRYTGVPSRFSGSGSGTDFTLTISSLQPEDFATYFCQQYSSYPYTFGGGTKVEIK

SEQ ID NO: 70 - protein, 107 aa, Artificial
  h011-3194-VL
DIVMTQSPSSLSASVGDRVTITCKASQNVYTAVAWYQQKPGKSPKLLIYSASNRYTGVPSRFSGSGSGTDFTLTISSLQPEDFATYFCQQLSSYPYTFGGGTKVEIK

SEQ ID NO: 71 - protein, 451 aa, Artificial
  h011-3133-IgG1
QVQLVQSGAEVKKPGASVKVSCKASGYTFTGYEIHWVRQAPGQGLEWMGYINPSATYTKFNQKFKDRVTMTRDTSISTAYMELSRLRSDDTAVYYCARERIYSNYWFFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 72 - DNA, 1413 bp, Artificial
  h011-3133-IgG1
atggagtttgggctgagctggctttttcttgtcgcgattcttaagggtgtccagtgccaggtgcagctggtgcagtctggcgccgaggtgaagaagcccggagcatctgtgaaggtgtcttgtaaggcctctggctatacctttaccggctacgagatccactgggtgcggcaggcacccgggcagggcctggagtggatgggctacatcaacccctctgctacctacaccaagtttaaccagaagttcaaggaccgggtgaccatgacccgggacacctctatctctaccgcctacatggagctgtctcggctgcggtctgacgacaccgccgtgtactactgcgcacgcgaacggatctactctaactactggttcttcgacgtgtggggccagggcaccaccgtgaccgtgtcttctgcttcgaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 73 - protein, 214 aa, Artificial
  h011-3133-kappa
DIVMTQSPSSLSASVGDRVTITCKASQNVYTAVAWYQQKPGKSPKLLIYSASNRYTGVPSRFSGSGSGTDFTLTISSLQPEDFATYFCQQYSSYPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 74 - DNA, 711 bp, Artificial
  h011-3133-kappa
atggacatgcgcgtgcccgcccagctgctgggcctgctgctgctgtggttccccggctcgcgatgcgacatcgtgatgacccagtctccctcatctctgagtgcctctgttggcgaccgggtgaccatcacctgcaaagcctctcagaacgtatacacagccgtggcctggtatcaacagaagcccggcaagtcccccaagctgctgatttactctgcctctaaccggtacaccggcgtgccctctcggttctctggctctggttctggcaccgacttcaccctgactatctcttctctgcagcccgaggacttcgccacctacttctgccagcagtactcttcttacccctacaccttcggcggaggcaccaaggtggagatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttga

SEQ ID NO: 75 - protein, 451 aa, Artificial
  h011-3133-IgG1-YTE
QVQLVQSGAEVKKPGASVKVSCKASGYTFTGYEIHWVRQAPGQGLEWMGYINPSATYTKFNQKFKDRVTMTRDTSISTAYMELSRLRSDDTAVYYCARERIYSNYWFFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 76 - DNA, 1413 bp, Artificial
  h011-3133-IgG1-YTE
atggagtttgggctgagctggctttttcttgtcgcgattcttaagggtgtccagtgccaggtgcagctggtgcagtctggcgccgaggtgaagaagcccggagcatctgtgaaggtgtcttgtaaggcctctggctatacctttaccggctacgagatccactgggtgcggcaggcacccgggcagggcctggagtggatgggctacatcaacccctctgctacctacaccaagtttaaccagaagttcaaggaccgggtgaccatgacccgggacacctctatctctaccgcctacatggagctgtctcggctgcggtctgacgacaccgccgtgtactactgcgcacgcgaacggatctactctaactactggttcttcgacgtgtggggccagggcaccaccgtgaccgtgtcttctgcttcgaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctctacatcacccgggagcctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

(19) United States
(12) Patent Application Publication
(10) Pub. No.: US 2020/0270365 A1
(43) Pub. Date: Aug. 27, 2020

(54) PCSK9 ANTIBODY, ANTIGEN-BINDING FRAGMENT THEREOF, AND MEDICAL USES THEREOF

(71) Applicants: Jiangsu Hengrui Medicine Co., Ltd., Lianyungang, Jiangsu (CN); Shanghai Hengrui Pharmaceutical Co., Ltd., Shanghai (CN)

(72) Inventors: Xiangdong QU, Shanghai (CN); Xin YE, Shanghai (CN); Houcong JIN, Shanghai (CN); Dongbing CUI, Shanghai (CN); Qiyue HU, Shanghai (CN); Weikang TAO, Shanghai (CN); Lianshan ZHANG, Shanghai (CN); Piaoyang SUN, Lianyungang, Jiangsu (CN)

(21) Appl. No.: 16/067,951

(22) PCT Filed: Dec. 26, 2016
(86) PCT No.: PCT/CN2016/112075

§ 371 Date: Jul. 3, 2018

(30) Foreign Application Priority Data
Jan. 5, 2016 (CN) 201610010241.3

(51) Int. Cl. C07K 16/40; A61P 3/06

(52) U.S. Cl. CPC C07K 16/40; A61K 2039/505; A61P 3/06

(57) ABSTRACT

The present invention provides a PCSK9 antibody, an antigen-binding fragment thereof, and medical uses thereof.

### CROSS REFERENCE TO RELATED APPLICATIONS

This application is a Section 371 of International Application No. PCT/CN2016/112075, filed Dec. 26, 2016, which was published in the Chinese Language on Jul. 13, 2017, under International Publication No. WO 2017/118307 A1, which claims priority to Chinese Patent Application No. 201610010241.3, filed on Jan. 5, 2016. Each disclosure is incorporated herein by reference in its entirety.

### REFERENCE TO SEQUENCE LISTING SUBMITTED ELECTRONICALLY

This application contains a sequence listing, which is submitted electronically via EFS-Web as an ASCII formatted sequence listing with a file name “688452_76US Sequence Listing” and a creation date of Jul. 2, 2018, and having a size of 99 kb. The sequence listing submitted via EFS-Web is part of the specification and is herein incorporated by reference in its entirety.

### FIELD OF THE INVENTION

The present invention relates to a PCSK9 antibody, an antigen-binding fragment thereof, a chimeric antibody, a humanized antibody comprising CDR regions of the PCSK9 antibody, and a pharmaceutical composition comprising the PCSK9 antibody and the antigen-binding fragment thereof, as well as its use as a medicament for lowering the level of blood lipid.

### BACKGROUND OF THE INVENTION

Hypercholesterolemia is a disease with abnormal metabolism of lipid, characterized in an increased level of serum cholesterol. Its main manifestation is the increased level of serum cholesterol, which causes cholesterol aggregated in vessels and consequently results in atherosclerosis formed. Abundant clinical and experimental research results have demonstrated that the abnormal metabolism of lipid is closely correlated with occurrence and development of coronary heart disease. Therefore, reducing the concentration of cholesterol in blood becomes a main means for treating and preventing atherosclerosis.

With the rapid improvement of the national standard of living in China, dyslipidemia is becoming a main factor endangering urban and rural residents of China. According to the statistic results in 2012, about 40% of deaths per year in China were attributed to cardiovascular disease. The morbidity of dyslipidemia in adults in China is 18.6%, and it is estimated now that 160 million of people have dyslipidemia. The morbidities of different types of dyslipidemia are as follows: 2.9% for hypercholesterolemia, 11.9% for hypertriglyceridemia, 7.4% for low, high density lipoproteinemia, and 3.9% for marginally increased blood cholesterol level. It was mentioned that there are 33 million of people having hypercholesterolemia in China, however, for local areas, the morbidity of dyslipidemia is far more serious than the above data, Chronic Disease Prevention and Control China Expert Consensus, by Chronic Disease Prevention and Control Branch from Disease Prevention and Control Committee, Ministry of Public Health, 2012.

At present, the medicaments clinically used for controlling lipid levels are mainly focused on statins. Lipitor, as a most widely used and a best-selling cholesterol-lowering medicament, reduces the production of cholesterol by blocking the effect of cholesterol-producing enzyme in liver, and therefore increases the uptake of cholesterol from blood into liver, so that reduces the concentration of cholesterol in blood. However, Lipitor has some disadvantages. Firstly, it will be understood from data, that Lipitor can reduce low density lipoprotein by 30% to 40%, however, an effectively reduced blood lipid level still cannot be achieved in many patients (low density lipoprotein<50 mg/dL). Secondly, there is racial difference among patients in response rate to Lipitor. Because of these reasons, the patients need a more effective medicine for reducing blood lipid.

Familial hypercholesterolemia (FM) is an autosomal single-gene dominant hereditary disease, the clinical features of which are significantly increased total cholesterol (TC) and low density lipoprotein-cholesterol (LDL-c) in blood, xanthelasmata, corneal arcus and premature cardiovascular disease. Mutation in low density lipoprotein receptor (LDL receptor, LDLR) gene causes LDLR deficiency or absence, consequently LDL-c will not be transported to liver to be eliminated, and hence the level of LDL-c in blood is increased. Currently it is clear that 3 genes are corrected with occurrence of FM, which are LDLR gene, apolipoprotein B100 gene and proprotein convertase subtilisin/kexin type 9 (PCSK9) gene, respectively.

Proprotein convertase subtilisin/kexin type 9 (PCSK9) is a proprotein convertase, which is a subfamily of protease K belonging to a secretory Bacillus subtilis family. The encoded protein is synthesized as a soluble proenzyme, and is intra-molecularly processed in the endoplasmic reticulum by self-catalyzing. According to experimental results, PCSK9 promotes degradation of LDLR and thus increases the amount of LDL cholesterol in plasma, while LDL receptor mediates endocytosis process of LDL in the liver, and the latter is a main pathway to remove LDL from the circulating system. Researchers have found that PCSK9 gene mutations were detected in 12.5% hypercholesterolemia (ADH) patients. There are various types of PCSK9 mutations. According to different influences of mutations on LDL-c level regulated by PCSK9, there are two types of mutations, including loss-of-function and gain-of-function. Loss-of-function mutations are associated with low blood cholesterol level and have effect on preventing occurrence of atherosclerotic heart disease. The rates of PCSK9 mutations associated with low cholesterol are higher in population of Africans than those in other races. PCSK9 gain-of-function mutations raise plasma cholesterol level by increasing the function of PCSK9 and reducing LDLR's expression, which will cause serious hypercholesterolemia and premature coronary atherosclerotic heart disease. It is found at present that PCSK9 gain-of-function mutations include D374Y, S127R, F216L, N157K, R306S and so on. In comparison with PCSK9 wild type, the LDLR on cell surface in D374Y mutant was decreased by 36%, and in S127R mutation was decreased by 10%.

As a potential new target, PCSK9 has become a hot topic in research of hypercholesterolemia. It is important for us to further understand the mechanism of cholesterol metabolism and find new therapeutic strategy. Many multinational pharmaceutical companies are developing monoclonal antibodies against PCSK9, which increase the concentration of LDLR on the liver surface and reduce the concentration of LDL in the blood by neutralizing PCSK9 in the blood. The relevant patents and patent applications are WO2011111007, WO2011072263, WO2013170367, WO2013169886, WO2013148284, WO2013091103, WO2013039958, WO2013039969, WO2013016648, WO2013008185, WO2012170607, WO2012168491, WO2012154999, WO2012109530, WO2012101251, WO2012088313, U.S. Pat. Nos. 8,829,165 B2, 8,030,457B2, 8,563,698B2, 8,859,741B2, 8,871,913B2, 8,871,914B2, 8,883,983B2, WO2012058137 and WO2012054438

This present invention provides PCSK9 antibodies with higher affinity, higher selectivity and higher bioactivity.

### SUMMARY OF THE INVENTION

The present invention provides a PCSK9 antibody specifically binding to PCSK9 or an antigen-binding fragment thereof, comprising a variable region containing at least one or more CDR regions selected from:

i) a HCDR1 of GYX¹IH (SEQ ID NO: 43), where X¹ is T, D or E;

ii) a HCDR2 of X²IX³PSX⁴TYTKFNQKFKD (SEQ ID NO: 44), wherein X² is Y or E; X³ is N, L, I or V; X⁴ is 5, G or A;

iii) a HCDR3 of AREX⁵IX⁶X⁷NYWFFDX⁸ (SEQ ID NO: 45), wherein X⁵ is R or N; X⁶ is Y or F; X⁷ is S or F; X⁸ is V or R;

iv) a LCDR1 of KASQNVYX₁X₂VX₃ (SEQ ID NO: 46), wherein X₁ is T or W; X₂ is A or E; X₃ is A, D or V;

v) a LCDR2 of X₄X₅X₆NRYT (SEQ ID NO: 47), wherein X₄ is S, E or Q; X₅ is A or M; X₆ is S or V; and

vi) a LCDR3 of QQX₇SX₈X₉PX₁₀T (SEQ ID NO: 48), wherein X₇ is Y, F or L; X₈ is S or W; X₉ is Y, F, Q or S; X₁₀ is Y, D or E.

In another preferred embodiment of the present invention, the PCSK9 antibody specifically binding to PCSK9 or the antigen-binding fragment thereof according to the present invention, comprises a HCDR1, a HCDR2 and a HCDR3 as shown in SEQ ID NO: 43, SEQ ID NO: 44 and SEQ ID NO: 45, respectively.

In another preferred embodiment of the present invention, the PCSK9 antibody specifically binding to PCSK9 or the antigen-binding fragment thereof according to the present invention, comprises a LCDR1, a LCDR2 and a LCDR3 as shown in SEQ ID NO: 46, SEQ ID NO: 47 and SEQ ID NO: 48, respectively.

Amino acid mutations can be made in the CDR regions of the above SEQ ID NOs: 43-48 according to the present invention by means of affinity maturation, so as to obtain higher activity.

In another preferred embodiment of the present invention, in the PCSK9 antibody specifically binding to PCSK9 or the antigen-binding fragment thereof according to the present invention, the HCDR1 is selected from the sequences of SEQ ID NO: 14, SEQ ID NO: 20 or SEQ ID NO: 21, or the sequences having at least 95% identity to the above sequences.

HCDR2 is selected from the sequences of SEQ ID NO: 15, SEQ ID NO: 22, SEQ ID NO: 23, SEQ ID NO: 24, SEQ ID NO: 25, SEQ ID NO: 26 or SEQ ID NO: 27, or the sequences having at least 95% identity to the above sequences;

HCDR3 is selected from the sequences of SEQ ID NO: 16, SEQ ID NO: 28, SEQ ID NO: 29 or SEQ ID NO: 30, or the sequences having at least 95% identity to the above sequences.

In another preferred embodiment of the present invention, in the PCSK9 antibody specifically binding to PCSK9 or the antigen-binding fragment thereof according to the present invention, the LCDR1 is selected from the sequences of SEQ ID NO: 17, SEQ ID NO: 31, SEQ ID NO: 32, SEQ ID NO: 33 or SEQ ID NO: 34, or the sequences having at least 95% identity to the above sequences; the LCDR2 is selected from the sequences of SEQ ID NO: 18, SEQ ID NO: 35, SEQ ID NO: 36 or SEQ ID NO: 37, or the sequences having at least 95% identity to the above sequences; the LCDR3 is selected from the sequences of SEQ ID NO: 19, SEQ ID NO: 38, SEQ ID NO: 39, SEQ ID NO: 40, SEQ ID NO: 41 or SEQ ID NO: 42, or the sequences having at least 95% identity to the above sequences.

In another preferred embodiment of the present invention, the PCSK9 antibody specifically binding to PCSK9 or the antigen-binding fragment thereof according to the present invention, comprises six CDR regions selected from:

1) Six CDR regions as shown in SEQ ID NO: 14, SEQ ID NO: 15, SEQ ID NO: 16, SEQ ID NO: 17, SEQ ID NO: 18 and SEQ ID NO: 19, respectively;

2) Six CDR regions as shown in SEQ ID NO: 14, SEQ ID NO: 22, SEQ ID NO: 16, SEQ ID NO: 17, SEQ ID NO: 18 and SEQ ID NO: 19, respectively;

3) Six CDR regions as shown in SEQ ID NO: 14, SEQ ID NO: 23, SEQ ID NO: 16, SEQ ID NO: 17, SEQ ID NO: 18 and SEQ ID NO: 19, respectively;

4) Six CDR regions as shown in SEQ ID NO: 20, SEQ ID NO: 15, SEQ ID NO: 16, SEQ ID NO: 17, SEQ ID NO: 18 and SEQ ID NO: 19, respectively;

5) Six CDR regions as shown in SEQ ID NO: 14, SEQ ID NO: 15, SEQ ID NO: 28, SEQ ID NO: 17, SEQ ID NO: 18 and SEQ ID NO: 19, respectively;

6) Six CDR regions as shown in SEQ ID NO: 14, SEQ ID NO: 15, SEQ ID NO: 29, SEQ ID NO: 17, SEQ ID NO: 18 and SEQ ID NO: 19, respectively;

7) Six CDR regions as shown in SEQ ID NO: 14, SEQ ID NO: 15, SEQ ID NO: 16, SEQ ID NO: 17, SEQ ID NO: 35 and SEQ ID NO: 19, respectively;

8) Six CDR regions as shown in SEQ ID NO: 14, SEQ ID NO: 15, SEQ ID NO: 16, SEQ ID NO: 17, SEQ ID NO: 36 and SEQ ID NO: 19, respectively;

9) Six CDR regions as shown in SEQ ID NO: 14, SEQ ID NO: 15, SEQ ID NO: 16, SEQ ID NO: 31, SEQ ID NO: 18 and SEQ ID NO: 19, respectively;

10) Six CDR regions as shown in SEQ ID NO: 14, SEQ ID NO: 15, SEQ ID NO: 16, SEQ ID NO: 32, SEQ ID NO: 18 and SEQ ID NO: 19, respectively;

11) Six CDR regions as shown in SEQ ID NO: 14, SEQ ID NO: 15, SEQ ID NO: 16, SEQ ID NO: 17, SEQ ID NO: 18 and SEQ ID NO: 38, respectively;

12) Six CDR regions as shown in SEQ ID NO: 14, SEQ ID NO: 15, SEQ ID NO: 16, SEQ ID NO: 17, SEQ ID NO: 18 and SEQ ID NO: 39, respectively;

13) Six CDR regions as shown in SEQ ID NO: 21, SEQ ID NO: 24, SEQ ID NO: 16, SEQ ID NO: 17, SEQ ID NO: 18 and SEQ ID NO: 19, respectively;

14) Six CDR regions as shown in SEQ ID NO: 14, SEQ ID NO: 25, SEQ ID NO: 16, SEQ ID NO: 17, SEQ ID NO: 18 and SEQ ID NO: 19, respectively;

15) Six CDR regions as shown in SEQ ID NO: 14, SEQ ID NO: 15, SEQ ID NO: 30, SEQ ID NO: 17, SEQ ID NO: 37 and SEQ ID NO: 19, respectively;

16) Six CDR regions as shown in SEQ ID NO: 14, SEQ ID NO: 15, SEQ ID NO: 16, SEQ ID NO: 17, SEQ ID NO: 18 and SEQ ID NO: 40, respectively;

17) Six CDR regions as shown in SEQ ID NO: 14, SEQ ID NO: 15, SEQ ID NO: 16, SEQ ID NO: 17, SEQ ID NO: 18 and SEQ ID NO: 41, respectively;

18) Six CDR regions as shown in SEQ ID NO: 14, SEQ ID NO: 26, SEQ ID NO: 16, SEQ ID NO: 17, SEQ ID NO: 18 and SEQ ID NO: 19, respectively;

19) Six CDR regions as shown in SEQ ID NO: 14, SEQ ID NO: 27, SEQ ID NO: 16, SEQ ID NO: 17, SEQ ID NO: 18 and SEQ ID NO: 19, respectively;

20) Six CDR regions as shown in SEQ ID NO: 14, SEQ ID NO: 15, SEQ ID NO: 16, SEQ ID NO: 33, SEQ ID NO: 18 and SEQ ID NO: 19, respectively;

21) Six CDR regions as shown in SEQ ID NO: 14, SEQ ID NO: 15, SEQ ID NO: 16, SEQ ID NO: 34, SEQ ID NO: 18 and SEQ ID NO: 19, respectively;

22) Six CDR regions as shown in SEQ ID NO: 14, SEQ ID NO: 15, SEQ ID NO: 16, SEQ ID NO: 17, SEQ ID NO: 18 and SEQ ID NO: 42, respectively; and

23) Six CDR regions as shown in SEQ ID NO: 14, SEQ ID NO: 15, SEQ ID NO: 30, SEQ ID NO: 17, SEQ ID NO: 18 and SEQ ID NO: 19, respectively.

In another preferred embodiment of the present invention, the PCSK9 antibody specifically binding to PCSK9 or the antigen-binding fragment thereof according to the present invention, is a murine-derived antibody or a fragment thereof.

In another preferred embodiment of the present invention, the PCSK9 antibody specifically binding to PCSK9 or the antigen-binding fragment thereof according to the present invention, is a chimeric antibody or a fragment thereof.

In another preferred embodiment of the present invention, the PCSK9 antibody specifically binding to PCSK9 or the antigen-binding fragment thereof according to the present invention, is a humanized antibody or a fragment thereof.

In another preferred embodiment of the present invention, with respect to the PCSK9 antibody specifically binding to PCSK9 or the antigen-binding fragment thereof according to the present invention, the heavy chain of the humanized antibody comprises a heavy chain variable region, wherein the FR sequence is derived from a combination sequence of human germline heavy chains IGHV1-2*02 and hjh6.1, or mutant sequences thereof; wherein the FR sequence comprises FR1, FR2, FR3 from IGHV1-2*02 and FR4 from hjh6.1, or a mutant sequence thereof, or comprises amino acid sequences having at least 95% identity to the above sequences.

In another preferred embodiment of the present invention, with respect to the PCSK9 antibody or the antigen-binding fragment thereof according to the present invention, the heavy chain sequence of the humanized antibody is a variant of the sequence set forth in SEQ ID NO: 10; wherein the variant preferably has 0-10 amino acid change(s) in the heavy chain variable region. The amino acid change may be a modification based on the technology available in the art for improving affinity or half-life of the antibody, for example, modifying the CDR amino acid sequences by using affinity maturation or modifying the FR amino acid sequences by using back-mutations.

In another preferred embodiment of the present invention, with respect to the PCSK9 antibody specifically binding to PCSK9 or the antigen-binding fragment thereof according to the present invention, the humanized antibody comprises a heavy chain FR region having 0-10 amino acid back-mutations, wherein the back-mutation is preferably selected from one or more back-mutations consisting of R72A, T74K, V68A, M70L, M48V, G49A, R67K and R38K.

In another preferred embodiment of the present invention, with respect to the PCSK9 antibody specifically binding to PCSK9 or the antigen-binding fragment thereof according to the present invention, the PCSK9 antibody comprises a VH, the amino acid sequence of which is selected from the group consisting of SEQ ID NO: 12, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57 and SEQ ID NO: 58, or selected from the sequences having at least 95% identity to the above sequences.

In another preferred embodiment of the present invention, with respect to the PCSK9 antibody or the antigen-binding fragment thereof according to the present invention, the light chain FR sequence of the humanized antibody light chain variable region is derived from the combination sequence of human germline light chains IGKV1-39*01 and hjk4.1, or the mutant sequences thereof. The light chain FR comprises FR1, FR2, FR3 from IGKV1-39*01 and FR4 from hjk4.1 or the mutant sequence thereof, or a sequence having at least 95% identity to the above sequences.

In another preferred embodiment of the present invention, with respect to the PCSK9 antibody specifically binding to PCSK9 or the antigen-binding fragment thereof according to the present invention, the humanized antibody comprises a light chain variable region of SEQ ID NO: 11 or a variant thereof; the variant means the presence of 1, 2, 3, 4, 5, 6, 7, 8, 9 or 10 amino acid changes in SEQ ID NO:11. The amino acid change may be a modification based on the technology available in the art for improving affinity or half-life of the antibody, for example, modifying the CDR amino acid sequences by using affinity maturation or modifying the FR amino acid sequences by using back-mutations.

In another preferred embodiment of the present invention, with respect to the PCSK9 antibody specifically binding to PCSK9 or the antigen-binding fragment thereof according to the present invention, the humanized antibody comprises a light chain FR region having 0-10 amino acid back-mutations, wherein the back-mutation is preferably selected from one or more amino acid back-mutation consisting of Q3V, A43S and Y87F.

In another preferred embodiment of the present invention, the PCSK9 antibody or the antigen-binding fragment thereof according to the present invention, comprises a VL, the amino acid sequence of which is selected from the group consisting of SEQ ID NO: 13, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69 and SEQ ID NO: 70, or selected from the sequences having at least 95% identity to the above sequences.

In another preferred embodiment of the present invention, the PCSK9 antibody specifically binding to PCSK9 or the antigen-binding fragment thereof according to the present invention, comprises a VH and a VL selected from the following groups:

1) the VH of SEQ ID NO: 12 and the VL of SEQ ID NO: 13;

2) the VH of SEQ ID NO: 49 and the VL of SEQ ID NO: 13;

3) the VH of SEQ ID NO: 50 and the VL of SEQ ID NO: 13;

4) the VH of SEQ ID NO: 51 and the VL of SEQ ID NO: 13;

5) the VH of SEQ ID NO: 52 and the VL of SEQ ID NO: 13;

6) the VH of SEQ ID NO: 53 and the VL of SEQ ID NO: 13;

7) the VH of SEQ ID NO: 12 and the VL of SEQ ID NO: 59;

8) the VH of SEQ ID NO: 12 and the VL of SEQ ID NO: 60;

9) the VH of SEQ ID NO: 12 and the VL of SEQ ID NO: 61;

10) the VH of SEQ ID NO: 12 and the VL of SEQ ID NO: 62;

11) the VH of SEQ ID NO: 12 and the VL of SEQ ID NO: 63;

12) the VH of SEQ ID NO: 12 and the VL of SEQ ID NO: 64;

13) the VH of SEQ ID NO: 54 and the VL of SEQ ID NO: 13;

14) the VH of SEQ ID NO: 55 and the VL of SEQ ID NO: 13;

15) the VH of SEQ ID NO: 56 and the VL of SEQ ID NO: 65;

16) the VH of SEQ ID NO: 12 and the VL of SEQ ID NO: 66;

17) the VH of SEQ ID NO: 12 and the VL of SEQ ID NO: 67;

18) the VH of SEQ ID NO: 57 and the VL of SEQ ID NO: 13;

19) the VH of SEQ ID NO: 58 and the VL of SEQ ID NO: 13;

20) the VH of SEQ ID NO: 12 and the VL of SEQ ID NO: 68;

21) the VH of SEQ ID NO: 12 and the VL of SEQ ID NO: 69;

22) the VH of SEQ ID NO: 12 and the VL of SEQ ID NO: 70; and

23) the VH of SEQ ID NO: 56 and the VL of SEQ ID NO: 13.

In another preferred embodiment of the present invention, with respect to the PCSK9 antibody or the antigen-binding fragment thereof according to the present invention, the heavy chain of a chimeric antibody or humanized antibody further comprises a heavy chain constant region derived from human IgG1, IgG2, IgG3 or IgG4 or a variant thereof, or a sequence having at least 95% identity to sequences thereof; preferably comprises a heavy chain constant region derived from human IgG1, IgG2, or IgG4 or a heavy chain constant region of IgG1, IgG2, or IgG4 variants wherein the amino acid mutations prolong the half-life of the antibody in the serum, most preferably comprises IgG1, IgG2, or IgG4 heavy chain constant region introduced with a YTE mutation.

The light chain of the chimeric antibody or humanized antibody further comprises a constant region derived from human κ chain, human λ chain or a variant thereof, or a sequence having at least 95% identity to sequences thereof.

In another preferred embodiment of the present invention, the PCSK9 antibody specifically binding to PCSK9 or the antigen-binding fragment thereof according to the present invention, comprises:

1) a heavy chain sequence of SEQ ID NO: 71 and a light chain sequence of SEQ ID NO: 73; or

2) a heavy chain sequence of SEQ ID NO: 75 and a light chain sequence of SEQ ID NO: 73.

On the other hand, the present invention provides a PCSK9 antibody or an antigen-binding fragment thereof, comprising one or more CDRs selected from a HCDR as shown in SEQ ID NO: 14, SEQ ID NO: 15 or SEQ ID NO: 16, or a HCDR having at least 95% identity to SEQ ID NO: 14, SEQ ID NO: 15 or SEQ ID NO: 16; and a LCDR as shown in SEQ ID NO: 17, SEQ ID NO:18 or SEQ ID NO: 19, or a LCDR having at least 95% identity to SEQ ID NO: 17, SEQ ID NO:18 or SEQ ID NO: 19. The sequence having at least 95% identity can be obtained by affinity maturation of the CDR region, such as the HCDR1 selected from SEQ ID NO: 20 and SEQ ID NO: 21, or the HCDR2 selected from SEQ ID NOs: 22-27, or the HCDR3 selected from SEQ ID NOs: 28-30; or the LCDR1 selected from SEQ ID NOs: 31-34, or the LCDR2 selected from SEQ ID NOs: 35-37, or the LCDR3 selected from SEQ ID NOs: 38-42. The HCDRs of present invention preferably comprise SEQ ID NO: 21, SEQ ID NO: 24 and SEQ ID NO: 16, and preferably the LCDRs comprise SEQ ID NO: 17, SEQ ID NO: 18 and SEQ ID NO: 19.

The present invention further provides a nucleic acid molecule encoding the PCSK9 antibody or the antigen-binding fragment thereof as described above.

The present invention further provides an expression vector comprising the nucleic acid molecule as described above.

The present invention further provides a host cell transformed with the expression vector as described above, wherein the host cell is selected from the group consisting of a prokaryotic cell and a eukaryotic cell, preferably a eukaryotic cell, more preferably a mammalian cell.

The present invention further provides a method for preparing a PCSK9 antibody, comprising culturing the host cell as described above under the conditions appropriate for expressing a nucleic acid encoding the PCSK9 antibody as described above, and/or recovering the PCSK9 antibody from the host cell.

The present invention further provides a pharmaceutical composition, comprising a therapeutically effective amount of the PCSK9 antibody or the antigen-binding fragment thereof as described above, and one or more pharmaceutically acceptable carriers, diluents or excipients.

The present invention further provides use of the PCSK9 antibody or the antigen-binding fragment thereof, or the pharmaceutical composition as described above, in preparation of a medicament for the treatment of a disease or a condition mediated by PCSK9, wherein the disease or the condition is preferably cholesterol related diseases (including “serum cholesterol related diseases”); the disease or the condition is more preferably selected from the group consisting of hypercholesterolemia, heart disease, metabolic syndrome, diabetes, coronary heart disease, stroke, cardiovascular disease, Alzheimer's disease and general dyslipidemia; most preferably selected from hypercholesterolemia, dyslipidemia, atherosclerosis, CVD or coronary heart disease.

The exemplary diseases which can be diagnosed with the antibody according to the present invention include cholesterol related diseases (including “serum cholesterol related diseases”), including any one or more disease selected from the group consisting of hypercholesterolemia, heart disease, metabolic syndrome, diabetes, coronary heart disease, stroke, cardiovascular disease, Alzhemier's disease and general dyslipidemia (characterized in an increased level of total serum cholesterol, LDL, triglyceride, very low density lipoprotein (VLDL) and/or a decreased level of HDL).

On one hand, the present invention provides a method of treating or preventing hypercholesterolemia and/or at least one symptom selected from dyslipidemia, atherosclerosis, cardiovascular disease (CVD) or coronary heart disease in an individual, wherein the method comprises administrating an effective amount of PCSK9 antibody to the individual. The present invention also provides use of an effective amount of PCSK9 antibody against extracellular or circulating PCSK9 in preparation of a medicament, wherein the medicament is for treating or preventing hypercholesterolemia and/or at least one symptom selected from dyslipidemia, atherosclerosis, CVD or coronary heart disease in an individual.

Any embodiment or combination thereof described in the present invention is suitable for any and all PCSK9 antibodies, methods and uses according to the present invention.

### BRIEF DESCRIPTION OF THE DRAWINGS

FIG. 1: Changes in LDL uptake by HepG2 cells under various concentrations of PCSK9 antibodies (h011-3058, h011-3065, h011-3133, h011-3147, h011-3191). The results show that PCSK9 antibodies can promote LDL uptake by HepG2 cells.

FIG. 2: Changes in LDL uptake by HepG2 cells under various concentrations of PCSK9 antibodies (h011-3050, h011-3095, h011-3181, h011-3187, h011-3190). The results show that PCSK9 antibodies can promote LDL uptake by HepG2 cells.

FIG. 3: Changes in serum concentrations of LDL-c after injection with various PCSK9 antibodies (h011-3133-WT, h011-3133-YTE, h011-3191, h011-3065) in mice (*:p<0.05, vs IgG, **:p<0.01, vs IgG). The results show that PCSK9 antibodies can reduce serum concentration of LDL-c in mice overexpressing human PCSK9.

FIG. 4: Changes in serum concentrations of LDL-c after injection with various PCSK9 antibodies (h011-3058, h011-3191, h011-3147) in mice (*:p<0.05, vs IgG, **:p<0.01, vs IgG). The results show that PCSK9 antibodies can reduce serum concentration of LDL-c in mice overexpressing human PCSK9.

FIG. 5: Changes in relative serum concentrations of LDL-c (vs that in IgG group) after injection with various PCSK9 antibodies (h011-3133-WT, h011-3133-YTE, h011-3191) in mice. The results show that compared to the IgG group, PCSK9 antibodies can reduce serum concentration of LDL-c in mice overexpressing human PCSK9.

FIG. 6: Changes in relative serum concentrations of LDL-c (vs that in IgG group) after injection with various PCSK9 antibodies (h011-3058, h011-3191, h011-3147) in mice. The results show that compared to the IgG group, PCSK9 antibodies can reduce serum concentration of LDL-c in mice overexpressing human PCSK9.

### DETAILED DESCRIPTION OF THE INVENTION

The description or technology and method of the present invention are generally well understood and usually used by those skilled artisans in the art, such as those widely used methods described in the following literatures: Sambrook et al., Molecular Cloning: A Laboratory Manual version 3 (2001); Cold Spring Harbor Laboratory Press, Cold Spring Harbor, N.Y. CURRENT PROTOCOL SINMOLECULAR BIOLOGY (The latest experimental method in molecular biology) (F. M. Ausubel et al., Ed., (2003)); METHOD SINENZYMOLOGY (Academic Press, Inc.): PCR2: A PRACTICAL APPROACH (PCR2: practical method) (M. J. MacPherson, B. D. Hames and G. R. Taylor Ed. (1995)), Harlow and Lane Ed. (1988); ANTIBODIES, A LABORATORY MANUAL and ANIMAL CELL CULTURE (R. I. Freshney Ed. (1987)); Oligonucleotide Synthesis (M. J. Gait Ed. 1984); Methods in Molecular Biology, Humana Press; Cell Biology: A Laboratory Notebook (J. E. C ellis Ed. 1998) Academic Press; Animal Cell Culture (R. I. Freshney) Ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths and D. G. Newell Ed., 1993-8) J. Wiley and Sons; Handbook of Experimental Immunology (D. M. Weir and C. C. Blackwell Ed.); Gene Transfer Vectors for Mammalian Cells (J. M. Miller and M. P. Calos Ed., 1987); PCR: The Polymerase Chain Reaction (Mullis et al., Ed. 1994); Current Protocols in Immunology (J. E. Coligan er al., Ed., 1991); Short Protocols in Molecular Biology (Wiley and Sons, 1999); Immunobiology (C. A. Janeway and P. Travers, 1997); Antibodies (P. Finch, 1997); Antibodies: A Practical Approach (D. Catty Ed., IRL Press, 1988-1989); Monoclonal Antibodies: A Practical Approach (P. Shepherd and C. Dean Ed., Oxford University Press, 2000); Using Antibodies: A Laboratory Manual (E. Harlow and D. Lane (Cold Spring Harbor Laboratory Press, 1999); The Antibodies (M. Zanetti and J. D. Capra Ed., Harwood Academic Publishers, 1995); and Cancer: Principles and Practice of Oncology (V. T. DeVita et al., Ed., J.B. Lippincott Company, 1993).

### DEFINITION

Unless specifically defined elsewhere in this document, all the technical and scientific terms used herein have the same meaning as commonly understood by one of ordinary skill in the art to which this invention belongs. Singleton et al., Dictionary of Microbiology and Molecular Biology, version 2. J. Wiley&Sons (New York, N.Y. 1994), and March, Advanced Organic Chemistry Reactions, Mechanisms and Structure, version 4, John Wiley&Sons (New York, N.Y. 1992). Many terms used in the present invention provide some guidance to those skilled artisans in the art. All references cited herein (including patent applications and publications) are completely incorporated by reference.

For the purposes of interpreting this specification, the following definitions will be used, and where appropriate, terms used in the singular may also include the plural and vice versa. It should be understood that the terminology used herein is for the purpose of describing particular embodiments only and is not intended to be limiting. In the event that any of the definitions described below conflict with any of the documents incorporated by reference herein, the definitions described below shall prevail.

The term “Proprotein convertase subtilisin/kexin type 9 (PCSK9)”, “PCSK9” or “NARC-1” as used herein, refers to any native PCSK9 from any vertebrate source, including mammals such as primates (e.g. humans) and rodents (e.g., mice and rats), unless otherwise indicated. The term encompasses “full-length,” unprocessed PCSK9 as well as any form of PCSK9 produced or processed by the cells. The term also encompasses naturally occurring variants of PCSK9, e.g., splice variants or allelic variants.

In this specification and the claims, the numbering of residues in the heavy chain of an immunoglobulin is according to EU index as described in Kabat et al., Sequences of Proteins of Immunological Interest, 5th Ed. Public Health Service, National Institutes of Health, Bethesda, Md., 1991. The above literature is incorporated herein by reference. “The EU index as in Kabat” refers to the numbering of residues in the human IgG1 EU antibody.

The term “variable region” or “variable domain” refers to the antibody heavy or light chain domain that is involved in binding of the antibody to an antigen. The variable domains of the heavy chain and light chain (VH and VL, respectively) of a native antibody generally have similar structures, wherein each domain comprises four conserved framework regions (FRs) and three hypervariable regions (CDRs). (See, e.g., Kindt Kuby et al. Immunology, 6th ed., W.H. Freeman and Co., page 91 (2007).) A single VH or VL domain may be sufficient to confer antigen-binding specificity. Furthermore, antibodies binding to a particular antigen may be isolated by using a VH or VL domain from an antibody binding to the antigen to screen a library of complementary VL or VH domains, respectively. See, e.g., Portolano et al., J. Immunol. 150:880-887 (1993); Clarkson et al, Nature 352:624-628 (1991).

The term “hypervariable region” or “HVR,” as used herein, refers to each region of an antibody variable domain which are hypervariable in sequence and/or form structurally defined loops (“hypervariable loops”). Generally, native four-chain antibodies comprise six CDRs; three within the VH (HI, H2, H3), and three within the VL (LI, L2, L3). CDRs generally comprise amino acid residues from the hypervariable loops and/or from the “complementarity determining regions” (CDRs), the latter being of highest sequence variability and/or involved in antigen recognition. Exemplary hypervariable loops occur at amino acid residues 26-32 (LI), 50-52 (L2), 91-96 (L3), 26-32 (HI), 53-55 (H2), and 96-101 (H3) (Chothia and Lesk, J. Mol. Biol. 196:901-917 (1987)). Exemplary CDRs (LCDR1, LCDR2, LCDR3, HCDR1, HCDR2, and HCDR3) occur at amino acid residues 24-34 of LI, 50-56 of L2, 89-97 of L3, 31-35B of H1, 50-65 of H2, and 95-102 of H3. (Kabat et al, Sequences of Proteins of Immunological Interest, 5th Ed. Public Health Service, National Institutes of Health, Bethesda, Md. (1991)). With the exception of CDR1 in VH, CDRs generally comprise the amino acid residues that form the hypervariable loops. CDRs also comprise “specificity determining residues,” or “SDRs,” which are residues that contact with the antigen. SDRs are contained within a CDR region referred to as truncated-CDR, or a-CDR. Exemplary a-CDRs (a-LCDR1, a-LCDR2, a-LCDR3, a-HCDR1, a-HCDR2, and a-HCDR3) occur at amino acid residues 31-34 of LI, 50-55 of L2, 89-96 of L3, 31-35B of H1, 50-58 of H2, and 95-102 of H3. (See Almagro and Fransson, Front. Biosci. 13: 1619-1633 (2008)). Unless otherwise indicated, CDR residues and other residues in the variable domain (e.g., FR residues) are numbered herein according to Kabat et al., supra.

The terms “anti-PCSK9 antibody”, “anti-PCSK9”, “PCSK9 antibody” or “antibody binding to PCSK9” are used interchangeably herein, and refer to an antibody capable of binding to PCSK9 with sufficient affinity and capable of being used as a diagnostic and/or therapeutic agent targeting PCSK9.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

The terms “full length antibody,” “intact antibody,” and “whole antibody” are used herein interchangeably to refer to an antibody having a structure substantially similar to a native antibody structure or having heavy chains that contain an Fc region as defined herein.

The antigen-binding fragment of the present invention is selected from a Fab, a Fab′-SH, a Fv, a scFv or (Fab′)₂ fragments.

An “antigen-binding fragment” refers to a molecule other than an intact antibody, and it comprises a portion of an intact antibody that binds to the antigen to which the intact antibody binds. Examples of antigen-binding fragment include but are not limited to Fv; Fab; Fab; Fab′-SH; F(ab′)2; diabodies; linear antibodies; single-chain antibody molecules (e.g. scFv); and multispecific antibodies formed by antigen-binding fragments. Digesting antibodies with papain will produce two identical antigen-binding fragments, referred to as “Fab” fragments, each of which has a single antigen-binding site, and a residual “Fc” fragment, the name reflects its ability to readily be crystallized. Pepsin treatment yields a F(ab′)2 fragment having two antigen-binding sites, which is still capable of cross-linking with an antigen.

Fab fragments also contain constant domains of the light chain and the first constant domain (CH1) of the heavy chain, in addition to heavy chain variable domains and light chain variable domains. Fab′ fragments differ from Fab fragments by the addition of a few residues, including one or more cysteine residues from the antibody hinge region, at the carboxyl terminus of the heavy chain CH1 domain. Fab′-SH is the designation herein for Fab′ in which the cysteine residue(s) within the constant domains carry a free thiol group. F(ab′)2 antibody fragment was originally produced as a pair of Fab′ fragments, wherein a hinge cysteine was located between the Fab′ fragments. Other chemical couplings of antibody fragments are also known.

The term “Fc region” herein is used to define a C-terminal region of the immunoglobulin heavy chain, wherein at least a portion of the constant region is contained. The term includes native Fc region and variant Fc region sequence. In one embodiment, human IgG heavy chain Fc region extends from Cys226, or from Pro230, to the carboxyl-terminus of the heavy chain. However, the C-terminal lysine (Lys447) may or may not be present within the Fc region. Unless otherwise specified herein, numbering of amino acid residues in the Fc region or constant region is according to the EU numbering system, also called as the EU index, as described in Kabat et al., Sequences of Proteins of Immunological Interest, 5th Ed. Public Health Service, National Institutes of Health, Bethesda, Md., 1991. Fc region is essential for the effector functions of antibodies. The effector functions include initiating complement-dependent cytotoxicity (CDC), initiating phagocytosis and antibody-dependent cell-mediated cytotoxicity (ADCC), and transferring antibodies across cellular barriers via transcytosis. In addition, Fc region is critical for maintaining the serum half-life of an antibody of class IgG (Ward and Ghetie, Ther. Immunol. 2:77-94 (1995)). Studies have found that the serum half-life of an IgG antibody is mediated by binding of Fc to the neonatal Fc receptor (FcRn). FcRn is a heterodimer consisting of a transmembrane a chain and a soluble β chain (β2-microglobulin). A method of producing an antibody with a decreased biological half-life by introducing mutations into the DNA fragment encoding the antibody was disclosed in U.S. Pat. No. 6,165,745. The mutations include amino acid substitutions at position 253, 310, 311, 433, or 434 within the Fc-hinge domain. A composition comprising a mutant IgG molecule was disclosed in U.S. Pat. No. 6,277,375 B1, the molecule has an increased serum half-life relative to the wild-type IgG, wherein the mutant IgG molecule comprises the following amino acid substitutions: threonine to leucine at position 252, threonine to serine at position 254, or threonine to phenylalanine at position 256 (T252L, T254S, or T256F). A mutant IgG with amino acid substitutions at position 433, 435, or 436 is also disclosed. An antibody variant comprising an IgG Fc region was disclosed in U.S. Pat. No. 6,528,624, the variant comprises amino acid substitutions at one or more of amino acid positions 270, 322, 326, 327, 329, 331, 333, and 334 within the human IgG Fc region. A modified IgG was disclosed in WO 02/060919 A2, the modified IgG comprises an IgG constant domain comprising one or more amino acid modifications relative to a wild-type IgG constant domain, wherein the modified IgG has an increased half-life compared to IgG having the wild-type IgG constant domain, and wherein the one or more amino acid modifications are located at one or more of positions 251, 253, 255, 285-290, 308-314, 385-389, and 428-435. Specifically, the “YTE” or “YTE mutation” described herein refers to a mutation combination within the Fc region of IgG1, for promoting the binding of the Fc region to human FcRn, prolonging the half-life of the antibody in human serum. The YTE mutant contains a combination of three “YTE mutations”: M252Y, S254T and T256E. Numbering of residues is according to EU Numbering System, which is also referred to as EU index (refer to U.S. Pat. No. 7,658,921), such as numbering of IgG heavy chains in Kabat et al. Compared to wild-type antibodies, YTE mutant antibodies have greatly prolonged half-life in serum, e.g., Dall'Acqua et al, J. Biol. Chem. 281: 23514-24 (2006) and U.S. Pat. No. 7,083,784.

“Fv” is a minimum antigen-binding fragment comprising a complete antigen-binding site. In one embodiment, a double-chain Fv consists of one heavy chain variable domain and one light chain variable domain tightly and non-covalently associated to form a dimer. In a single-chain Fv (scFv), one heavy chain variable domain and one light chain variable domain can be covalently linked via a flexible peptide linker such that the light chain and heavy chain can be associated in a “dimeric” structure similar to that of a double-chain Fv. It is in such configuration that the three CDRs of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) has the ability to recognize and bind to antigen, the affinity is lower than that of the entire binding site.

A “single-chain Fv” or “scFv” antigen-binding fragment comprises the VH and VL domains of an antibody, wherein these domains are present as a single polypeptide chain. Generally, the scFv polypeptide further comprises a polypeptide linker between the VH and VL domains which enables the scFv to form the desired structure for antigen binding. For a review of scFv, see Pluckthun, in The Pharmacology of Monoclonal Antibodies, vol. 113, Rosenburg and Moore ed., Springer-Verlag, New York, pp. 269-315 (1994).

The term “chimeric antibody”, is an antibody which is formed by fusing the variable region of a murine antibody with the constant region of human antibody, so as to alleviate the murine antibody-induced immune response. To establish a chimeric antibody, a hybridoma secreting specific murine monoclonal antibody is first established, variable region genes are then cloned from mouse hybridoma cells, and then constant region genes of human antibody are cloned as desired, the mouse variable region genes are ligated with human constant region genes to form a chimeric gene which can be inserted into a human vector, and finally the chimeric antibody molecule is expressed in a eukaryotic or prokaryotic industrial system. In a preferred embodiment of the present invention, the light chain of the PCSK9 chimeric antibody further comprises light chain Fc regions derived from human κ, λ chain or a variant thereof. The heavy chain of the PCSK9 chimeric antibody further comprises heavy chain Fc regions derived from human IgG1, IgG2, IgG3 or IgG4, or a variant thereof, preferably comprises heavy chain constant regions derived from human IgG1, IgG2, IgG3 or IgG4, or preferably comprises heavy chain constant regions derived from human IgG1, IgG2 or IgG4 variants with prolonged half-life in serum via amino acid mutations (e.g., YTE mutations).

A “human antibody” is an antibody having amino acid sequences corresponding to those of an antibody produced from a human or human cells or derived from non-human sources that utilize human antibody repertoires or other human antibody-encoding sequences. Such definition of a human antibody specifically excludes humanized antibody comprising non-human antigen-binding residues.

A “humanized” antibody refers to a chimeric antibody comprising amino acid residues from non-human CDRs and amino acid residues from human FRs. In certain embodiments, a humanized antibody will comprise substantially all of at least one, and typically two, variable domains, in which all or substantially all of the CDRs (e.g., CDRs) are corresponding to those of a non-human antibody, and all or substantially all of the FRs are corresponding to those of human antibody. A humanized antibody optionally can comprise at least a portion of an antibody constant region derived from a human antibody. A “humanized form” of an antibody, e.g., a non-human antibody, refers to an antibody which has been humanized.

“Framework” or “FR” refers to variable domain residues other than hypervariable region (HRV) residues. The FRs within variable domains generally consist of four FR domains: FR1, FR2, FR3, and FR4. Accordingly, the HRV and FR sequences generally appear in the following order within VH (or VL): FR1-H1(L1)-FR2-H2(L2)-FR3-H3(L3)-FR4.

“Human consensus framework” is a framework which represents the most commonly occurring amino acid residues in a selection of human immunoglobulin VL or VH framework sequences. Generally, human immunoglobulin VL or VH sequences are selected from a subtype of variable domain sequences. Generally, the subtype of sequences is a subtype as described in Kabat et al., Sequences of Proteins of Immunological Interest, Fifth Edition, NIH Publication 91-3242, Bethesda Md. (1991), vol. 1-3. In one embodiment, for the VL, the subtype is subtype kappa I as described in Kabat et al., supra. In one embodiment, for the VH, the subtype is subtype III as described in Kabat et al, supra.

“Acceptor human framework” for the purposes herein is a framework comprising the amino acid sequence of a light chain variable domain (VL) framework or a heavy chain variable domain (VH) framework derived from human immunoglobulin framework or human consensus framework, as defined below. An acceptor human framework “derived from” human immunoglobulin framework or human consensus framework may comprise the same amino acid sequence thereof, or it may contain amino acid sequence changes. In some embodiments, the number of amino acid changes are 10 or less, 9 or less, 8 or less, 7 or less, 6 or less, 5 or less, 4 or less, 3 or less, or 2 or less. In some embodiments, the VL acceptor human framework sequence is identical to the VL human immunoglobulin framework sequence or human consensus framework sequence.

“Affinity” refers to the strength of the sum total of non-covalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein. Specific illustrative and exemplary embodiments for measuring binding affinity are described below.

An “affinity matured” antibody is an antibody with one or more alterations in one or more CDRs thereof which result in an improvement in the affinity of the antibody for antigen, compared to a parent antibody which does not possess those alteration(s).

The terms “host cell,” “host cell line,” and “host cell culture” are used interchangeably and refer to cells into which exogenous nucleic acid has been introduced, including the progeny of such cells. Host cells include “transformants” and “transformed cells,” including primarily transformed cells and progeny derived therefrom, regardless of the number of passages. Progeny may not be completely identical in nucleic acid content to a parent cell, but may contain mutations. Mutant progenies that have the same function or biological activity as screened or selected for the originally transformed cells are included herein.

An “isolated” antibody is an antibody which has been separated from components in its natural environment. In some embodiments, an antibody is purified to greater than 95% or 99% purity as determined by, for example, electrophoretic (e.g., SDS-PAGE, isoelectric focusing (IEF), capillary electrophoresis) or chromatography (e.g., ion exchange or reverse phase HPLC). For review, methods for assessment of antibody purity can be found in, e.g., Flatman et al, J. Chromatogr. B 848:79-87 (2007).

“Isolated nucleic acid encoding anti-PCSK9 antibody” refers to one or more nucleic acid molecules encoding antibody heavy and light chains (or fragments thereof), including such nucleic acid molecule(s) in a single vector or separate vectors, and such nucleic acid molecule(s) present at one or more locations in a host cell.

The term “vector,” as used herein, refers to a nucleic acid molecule capable of propagating another nucleic acid to which it is linked. The term includes a vector as a self-replicating nucleic acid structure as well as a vector incorporated into the genome of a host cell into which it has been introduced. Certain vectors are capable of directing the expression of nucleic acids to which they are operatively linked. Such vectors are referred to herein as “expression vectors.”

The term “monoclonal antibody” as used herein refers to an antibody obtained from a population of substantially homogeneous antibodies, i.e., the individual antibodies comprising the population are identical and/or bind to the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be produced by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies are described herein.

“Naked antibody” refers to an antibody that is not conjugated to a heterologous moiety (e.g., a cytotoxic moiety) or radiolabel. A naked antibody may be present in a pharmaceutical formulation.

“Native antibody” refers to naturally occurring immunoglobulin molecule with varying structures. For example, native IgG antibody is a heterotetrameric glycoprotein having about 150,000 Daltons, it is composed of two identical light chains associated with two identical heavy chains via disulfide bond. Each heavy chain has variable regions (VH), also called as variable heavy domains or a heavy chain variable domains from N- to C-terminus, followed by three constant domains (CHI, CH2, and CH3). Similarly, each light chain has variable regions (VL), also called as variable light domains or a light chain variable domains from N- to C-terminus, followed by constant light (CL) domains. The light chain of an antibody may be assigned to one of two types, called as kappa (κ) and lambda (λ), based on the amino acid sequence of its constant domain.

“Percent (%) amino acid sequence identity” relative to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved by various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithm needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNLX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction of X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal to the % amino acid sequence identity of B to A. Unless specifically stated otherwise, all % amino acid sequence identity values used herein are obtained as described in the immediately preceding paragraph using the ALIGN-2 computer program.

An “effective amount” of an agent, e.g., a pharmaceutical formulation, refers to an amount effective, at dosages and for periods of time necessary, to achieve the desired therapeutic or prophylactic result.

The term “hypercholesterolemia,” as used herein, refers to a condition in which cholesterol levels are elevated above a desired level. In certain embodiments, the LDL-cholesterol level is elevated above the desired level. In certain embodiments, the serum LDL-cholesterol levels are elevated above the desired level.

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is human.

The term “pharmaceutical formulation” or “pharmaceutical composition” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient other than an active ingredient in a pharmaceutical formulation, which is nontoxic to a subject. A pharmaceutically acceptable carrier includes, but is not limited to, buffer, excipient, stabilizer, or preservative.

The term “PCSK9 activity” or “biological activity” of PCSK9, as used herein, includes any biological effect of PCSK9. In certain embodiments, PCSK9 activity includes the ability of PCSK9 to interact with or bind to a substrate or receptor. In certain embodiments, the biological activity of PCSK9 is the ability of PCSK9 to bind to a LDL-receptor (LDLR). In certain embodiments, PCSK9 activity includes the ability of PCSK9 to decrease or reduce the availability of LDLR. In certain embodiments, the biological activity of PCSK9 includes the ability of PCSK9 to increase the amount of LDL in a subject. In certain embodiments, the biological activity of PCSK9 includes the ability of PCSK9 to decrease the amount of LDLR that is available to bind to LDL in a subject. In certain embodiments, the biological activity of PCSK9 includes the ability of PCSK9 to decrease the amount of LDLR that is available to bind to LDL. In certain embodiments, biological activity of PCSK9 includes any biological activity resulting from PCSK9 signaling.

As used herein, “treatment” refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishing any direct or indirect pathological consequences of the disease, decreasing the rate of disease progression, amelioration or palliation of the disease state, and improving prognosis. In some embodiments, antibodies of the invention are used to delay development of a disease or to slow down the progression of a disease.

### Compositions and Methods

In one aspect, the present invention is based, in part, on the experimental results obtained by using PCSK9 antibody. Results obtained indicate that blocking biological activity of PCSK9 with anti-PCSK9 antibodies leads to a prevention of reduction in LDLR. In addition, the results demonstrate that administration of anti-PCSK9 antibody reduces total LDL-cholesterol level in a subject. Accordingly, PCSK9 antibodies of the invention, as described herein, provide important therapeutic and diagnostic agents for use in targeting pathological conditions associated with PCSK9, e.g., cholesterol related disorders.

In certain embodiments, “cholesterol related disorder” includes any one or more selected from: hypercholesterolemia, heart disease, metabolic syndrome, diabetes, coronary heart disease, stroke, cardiovascular diseases, Alzheimer's disease and general dyslipidemia, characterized in, for example, an elevated total serum cholesterol level, elevated LDL level, elevated triglycerides level, elevated VLDL level, and/or lowered HDL level. Some non-limiting examples of primary and secondary dyslipidemias which can be treated with an anti-PCSK9 antibody, either alone, or in combination with one or more other agents, include the metabolic syndrome, diabetes mellitus, familial combined hyperlipidemia, familial hypertriglyceridemia, familial hypercholesterolemia, including heterozygous hypercholesterolemia, homozygous hypercholesterolemia, familial defective apoplipoprotein B-100; polygenic hypercholesterolemia; remnant removal disease, hepatic lipase deficiency; dyslipidemia secondary to any of the following: dietary indiscretion, hypothyroidism, drugs including estrogen and progestin therapy, beta-blockers, and thiazide diuretics; nephrotic syndrome, chronic renal failure, Cushing's syndrome, primary biliary cirrhosis, glycogen storage diseases, hepatoma, cholestasis, acromegaly, insulinoma, isolated growth hormone deficiency, and alcohol-induced hypertriglyceridemia. Anti-PCSK9 antibodies described herein can also be useful in preventing or treating atherosclerotic diseases, such as, for example, coronary heart disease, coronary artery disease, peripheral arterial disease, stroke (ischemic and hemorrhagic), angina pectoris, or cerebrovascular disease and acute coronary syndrome, myocardial infarction. In certain embodiments, the anti-PCSK9 antibodies described herein are useful in reducing the risk of: nonfatal heart attacks, fatal and non-fatal stroke, certain types of heart surgery, hospitalization for heart failure, chest pain in patients with heart disease, and/or cardiovascular events due to established heart disease such as precedent heart attack, precedent heart surgery, and/or chest pain with evidence of clogged arteries. In certain embodiments, the anti-PCSK9 antibodies and methods described herein can be used to reduce the risk of recurrent cardiovascular events.

### Exemplary Anti-PCSK9 Antibodies

In one aspect, the invention provides an isolated antibody specifically binding to PCSK9. In certain embodiments, the anti-PCSK9 antibody activates the activity of PCSK9.

In some embodiments, the anti-PCSK9 antibody may be humanized. In one embodiment, the anti-PCSK9 antibody comprises CDRs as defined in any of the above embodiments, and further comprises acceptor human frameworks, e.g. human immunoglobulin frameworks or human consensus frameworks.

In another aspect, the anti-PCSK9 antibody comprises a heavy chain variable domain (VH) sequence having at least 90%, 91%, 92%, 93%, 94%, 95%, 96%, 97%, 98%, 99% or 100% sequence identity to the amino acid sequence selected from SEQ ID NOs: 12 and 49-58. In certain embodiments, the VH sequence having at least 90%, 91%, 92%, 93%, 94%, 95%, 96% o, 97%, 98% or 99% identity contains substitutions (e.g., conservative substitutions), insertions, or deletions relative to the reference sequence, but the anti-PCSK9 antibody comprising said sequence retains the ability to bind to PCSK9. In certain embodiments, a total of 1 to 10 amino acids have been substituted, inserted and/or deleted in SEQ ID NO: 12, 49-57 or 58. In certain embodiments, substitutions, insertions, or deletions occur in regions outside the CDRs (i.e., in the FRs). Optionally, the anti-PCSK9 antibody comprises a VH sequence as shown in SEQ ID NO: 12, 49-57 or 58, including post-translational modifications of said sequence. In a particular embodiment, the VH comprises at least one, two, or three CDRs selected from: (a) a HCDR1 comprising an amino acid sequence of SEQ ID NO: 14, 20 or 21; (b) a HCDR2 comprising an amino acid sequence of SEQ ID NO: 15, 22-26 or 27; and (c) a HCDR3 comprising an amino acid sequence of SEQ ID NO: 16, 28-29 or 30.

In another aspect, an anti-PCSK9 antibody is provided, wherein the antibody comprises a light chain variable domain (VL) having at least 90%, 91%, 92%, 93%, 94%, 95%, 96%, 97%, 98%, 99% or 100% sequence identity to the amino acid sequence of SEQ ID NO: 13, 59-69 or 70. In certain embodiments, the VL sequence having at least 90%, 91%, 92%, 93%, 94%, 95%, 96%, 97%, 98% or 99% identity contains substitutions (e.g., conservative substitutions), insertions, or deletions relative to the reference sequence, but an anti-PCSK9 antibody comprising said sequence retains the ability to bind to PCSK9. In certain embodiments, a total of 1 to 10 amino acids have been substituted, inserted and/or deleted in SEQ ID NO: 13, 59-69 or 70. In certain embodiments, the substitutions, insertions, or deletions occur in regions outside the CDRs (i.e., in the FRs). Optionally, the anti-PCSK9 antibody comprises a VL sequence as shown in SEQ ID NO: 13, 59-69 or 70, including post-translational modifications of said sequence. In a particular embodiment, the VL comprises at least one, two, or three CDRs selected from: (a) a LCDR1 comprising an amino acid sequence of SEQ ID NO: 17, 31-33 or 34; (b) a LCDR2 comprising an amino acid sequence of SEQ ID NO: 18, 35-36 or 37; and (c) a LCDR3 comprising an amino acid sequence of SEQ ID NO: 19, 38-41 or 42.

In another aspect, an anti-PCSK9 antibody is provided, wherein the antibody comprises a VH as described in any of the embodiments provided above, and a VL as described in any of the embodiments provided above. In one embodiment, the antibody comprises VH and VL sequences as shown in SEQ ID NO: 12 and SEQ ID NO: 13, respectively, including post-translational modifications of those sequences. In one embodiment, the antibody comprises VH and VL sequences as shown in SEQ ID NO: 12 and SEQ ID NO: 59, respectively, including post-translational modifications of those sequences. In one embodiment, the antibody comprises VH and VL sequences as shown in SEQ ID NO: 52 and SEQ ID NO: 13, respectively, including post-translational modifications of those sequences. In one embodiment, the antibody comprises VH and VL sequences as shown in SEQ ID NO: 54 and SEQ ID NO: 13, respectively, including post-translational modifications of those sequences. In one embodiment, the antibody comprises VH and VL sequences as shown in SEQ ID NO: 56 and SEQ ID NO: 65, respectively, including post-translational modifications of those sequences. In one embodiment, the antibody comprises VH and VL sequences as shown in SEQ ID NO: 56 and SEQ ID NO: 13, respectively, including post-translational modifications of those sequences.

In a further aspect of the invention, the anti-PCSK9 antibody according to any of the above embodiments is a monoclonal antibody, including a chimeric, humanized or human antibody. In one embodiment, the anti-PCSK9 antibody is an antigen-binding fragment, e.g., a Fv, a Fab, a Fab′, a scFv, a diabody, or a F(ab′)2 fragment. In another embodiment, the antibody is a full length antibody, e.g., an intact IgG1 antibody or other antibody class or isotype as defined herein.

In a further aspect, the anti-PCSK9 antibody according to any of the above embodiments may have any of the features, alone or in combination, as described in Sections below:

1. Antibody Affinity

In certain embodiments, the antibody provided herein has dissociation constant (Kd) of ≤1 μM, ≤100 nM, ≤10 nM, ≤1 nM, ≤0.1 nM, ≤0.01 nM, or ≤0.001 nM (e.g. 10E-8M or less, e.g. from 10E-8M to 10E-13M, e.g., from 10E-9M to 10E-13M).

2. Antigen-Binding Fragment

In certain embodiments, the antibody provided herein is an antigen-binding fragment. Antigen-binding fragments include, but are not limited to, Fab, Fab′, Fab′-SH, F(ab′)2, Fv, and scFv fragments, and other fragments described below. For review of certain antigen-binding fragments, see Hudson et al. Nat. Med. 9: 129-134 (2003). For review of scFv fragments, see, e.g., Pluckthun, The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458. For discussion of Fab and F(ab′)2 fragments comprising salvage receptor binding epitope residues and having increased in vivo half-life, see U.S. Pat. No. 5,869,046.

Single-domain antibodies are antigen-binding fragments comprising all or a portion of the heavy chain variable domain or all or a portion of the light chain variable domain of an antibody. In certain embodiments, a single-domain antibody is a human single-domain antibody (Domantis, Inc., Waltham, Mass.; see, e.g., U.S. Pat. No. 6,248,516 B1).

Antigen-binding fragments can be made by various techniques, including but not limited to proteolytic digestion of an intact antibody as well as production by recombinant host cells (e.g. E. coli or phage), as described herein.

3. Chimeric and Humanized Antibodies

In certain embodiments, the antibody provided herein is a chimeric antibody. Certain chimeric antibodies are described, e.g., in U.S. Pat. No. 4,816,567; and Morrison et al, Proc. Natl. Acad. Sci. USA, 81 :6851-6855 (1984)). In one example, a chimeric antibody comprises a non-human variable region (e.g., a variable region derived from mouse, rat, hamster, rabbit, or non-human primate, such as a monkey) and a human constant region. In a further example, a chimeric antibody is a “class switching” antibody in which the class or subclass has been changed from that of the parent antibody. Chimeric antibodies include antigen-binding fragments thereof.

In certain embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. The humanized antibody optionally will also comprise at least a portion of a human constant region. In some embodiments, some FR residues in a humanized antibody are substituted with corresponding residues from a non-human antibody (e.g., an antibody from which the CDR residues are derived), e.g., to restore or improve antibody specificity or affinity.

Humanized antibodies and methods of making the same are reviewed, e.g., in Almagro and Fransson, Front. Biosci. 13: 1619-1633 (2008), and are further described, e.g., in Riechmann et al, Nature 332:323-329 (1988); Queen et al, Proc. Nat'l Acad. Sci. USA 86: 10029-10033 (1989); U.S. Pat. Nos. 5,821,337, 7,527,791, 6,982,321, and 7,087,409; Kashmiri et al, Methods 36:25-34 (2005) (describing SDR (a-CDR) grafting); Padlan, Mol. Immunol. 28:489-498 (1991) (describing “resurfacing”); DaU'Acqua et al, Methods 36:43-60 (2005) (describing “FR shuffling”); and Osbourn et al, Methods 36:61-68 (2005) and Klimka et al, Br. J. Cancer, 83: 252-260 (2000) (describing the “guided selection” approach for FR shuffling).

Human framework regions that may be used for humanization include but are not limited to: framework regions selected with the “best-fit” method (see, e.g., Sims et al. J. Immunol. 151 :2296 (1993)); framework regions derived from the consensus sequence of human antibodies of a particular subtype of light or heavy chain variable regions (see, e.g., Carter et al. Proc. Natl. Acad. Sci. USA, 89:4285 (1992); and Presta et al. J. Immunol, 151 :2623 (1993)); human mature (somatically mutated) framework regions or human germline framework regions (see, e.g., Almagro and Fransson, Front. Biosci. 13: 1619-1633 (2008)); and framework regions derived from screening FR libraries (see, e.g., Baca et al, J. Biol. Chem. 272: 10678-10684 (1997) and Rosok et al, J. Biol. Chem. 271: 22611-22618 (1996)).

4. Library-Derived Antibodies

Antibodies of the invention may be isolated by screening combinatorial libraries for antibodies with the desired activity or activities. For example, a variety of methods are known in the art for generating phage display libraries and screening such libraries for antibodies possessing the desired binding characteristics. Such methods are reviewed, e.g., in Hoogenboom et al. Methods in Molecular Biology 178: 1-37 (O'Brien et al, ed., Human Press, Totowa, N.J., 2001) and further described, e.g., in McCafferty et al, Nature 348:552-554; Clackson et al, Nature 352: 624-628 (1991); Marks et al, J. Mol. Biol. 222: 581-597 (1992); Marks and Bradbury, Methods in Molecular Biology 248: 161-175 (Lo, ed., Human Press, Totowa, N.J., 2003); Sidhu et al, J. Mol. Biol. 338(2): 299-310 (2004); Lee et al, J. Mol. Biol. 340(5): 1073-1093 (2004); Fellouse, Proc. Natl. Acad. Sci. USA 101(34): 12467-12472 (2004); and Lee et al, J. Immunol. Methods 284(1-2): 119-132 (2004).

In certain phage display methods, repertoires of VH and VL genes are separately cloned by polymerase chain reaction (PCR) and recombined randomly in phage libraries, which can then be screened for antigen-binding phage as described in Winter et al., Ann. Rev. Immunol., 12: 433-455 (1994). Phage typically display antigen-binding fragments, either as single-chain Fv (scFv) fragments or as Fab fragments. Libraries from immunized sources provide high-affinity antibodies against the immunogen without the requirement of constructing hybridomas. Alternatively, the naive repertoire can be cloned (e.g., from human) to provide a single source of antibodies against a wide range of non-self and also self-antigens without any immunization as described by Griffiths et al, EMBO J 12: 725-734 (1993). Finally, naive libraries can also be made synthetically by cloning un-rearranged V-gene segments from stem cells, and using PCR primers containing random sequence to encode the highly variable CDR3 regions and to accomplish rearrangement in vitro, as described by Hoogenboom and Winter, J. Mol. Biol, 227: 381-388 (1992). Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360. Antibodies or antibody fragments isolated from human antibody libraries are considered as human antibodies or human antibody fragments herein.

5. Antibody Variants

In certain embodiments, amino acid sequence variants of the antibodies provided herein are contemplated. For example, it may be desirable to improve the binding affinity, prolong serum half-life and/or other biological properties of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

### Substitution, Insertion, and Deletion Variants

In certain embodiments, antibody variants having one or more amino acid substitutions are provided. Sites of interest for substitution mutagenesis include the CDRs and FRs. Conservative substitutions are shown in Table 1 under the heading of “conservative substitutions.” More substantial changes are provided in Table 1 under the heading of “exemplary substitutions” and as further described below in reference to amino acid side chain classes. Amino acid substitutions may be introduced into an antibody of interest and the products are screened for a desired activity, e.g., retained/improved antigen binding, decreased immunogenicity, or prolonged half-life products.

TABLE 1

| Original Residue | Exemplary Substitutions | Preferred Substitutions |
|---|---|---|
| Ala (A) | Val; Leu; Ile | Val |
| Arg (R) | Lys; Gln; Asn | Lys |
| Asn (N) | Gln; His; Asp; Lys; Arg | Gln |
| Asp (D) | Glu; Asn | Glu |
| Cys (C) | Ser; Ala | Ser |
| Glv (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; | Leu |
| | Nle | |
| Leu (L) | Nle; Ile; Val; Met; Ala; | Ile |
| | Phe | |
| Lys (K) | Arg; Gln; Asn | Arg |
| Met (M) | Leu; Phe; Ile | Leu |
| Phe (F) | Trp; Leu; Val; Ile; Ala; | Try |
| | Tyr | |
| Pro (p) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; | Leu |
| | Nle | |

Amino acids may be grouped according to common side-chain properties:

(1) Hydrophobic: Norleucine (Nle), Met, Ala, Val, Leu, Ile;

(2) Neutral hydrophilic: Cys, Ser, Thr, Asn, Gln;

(3) Acidic: Asp, Glu;

(4) Basic: His, Lys, Arg;

(5) Residues that influence chain orientation: Gly, Pro;

(6) Aromatic: Trp, Tyr, Phe;

Non-conservative substitutions will entail exchanging a member of one of these classes for another class.

One type of substitution variant involves substituting one or more hypervariable region residues of a parent antibody (e.g. humanized or human antibody). Generally, the resulting variant(s) selected for further study will have modifications (e.g., improvements) in certain biological properties (e.g., increased affinity, prolonged half-life or reduced immunogenicity) relative to the parent antibody and/or will have substantially retained certain biological properties of the parent antibody. An exemplary substitution variant is an affinity matured antibody, which may be conveniently generated, e.g., using phage display-based affinity maturation techniques such as those described herein. Briefly, one or more CDR residues are mutated and the variant antibodies will be displayed on phage and screened for a particular biological activity (e.g. binding affinity).

Alterations (e.g., substitutions) may be made in CDRs, e.g., to improve antibody affinity. Such alterations may be made in CDR “hotspots,” i.e., residues encoded by codons that undergo mutations at high frequency during the somatic maturation process (see, e.g., Chowdhury, Methods Mol. Biol. 207: 179-196 (2008)), and/or SDRs (a-CDRs), with the resulting variant VH or VL being tested for binding affinity. Affinity maturation obtained by constructing and reselecting from secondary libraries has been described, e.g., in Hoogenboom et al. Methods in Molecular Biology 178: 1-37 (O'Brien et al, ed., Human Press, Totowa, N.J., (2001)). In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variant with the desired affinity. Another method to introduce diversity involves CDR-directed approaches, in which several CDR residues (e.g., 4-6 residues at a time) are randomized. CDR residues involved in antigen binding may be specifically identified, e.g., using alanine scanning mutagenesis or modeling. CDR-H3 and CDR-L3 in particular are often targeted.

In certain embodiments, substitutions, insertions, or deletions may occur within one or more CDRs so long as such alterations do not substantially reduce the ability of the antibody to bind to an antigen. For example, conservative alterations (e.g., conservative substitutions as provided herein) that do not substantially reduce binding affinity may be made in CDRs. Such alterations may be outside of CDR “hotspots” or SDRs. In certain embodiments of the variant VH and VL sequences provided above, each CDR either is unaltered, or contains no more than one, two or three amino acid substitutions.

### Fc Region Variants

In certain embodiments, one or more amino acid modifications may be introduced into the Fc region of an antibody provided herein, thereby generating an Fc region variant and promoting the binding of the Fc region to human FcRn, and the half-life of the antibody in human serum is prolonged. The Fc region variant may comprise human Fc region sequence {e.g., human IgG1, IgG2, IgG3 or IgG4 Fc region) comprising amino acid modifications {e.g. substitutions) at one or more amino acid positions.

### Assays

Anti-PCSK9 antibodies provided herein may be identified, screened for, or characterized for their physical/chemical properties and/or biological activities by various assays known in the art.

1. Binding Assays and Other Assays

In one aspect, anti-PCSK9 antibodies of the invention are tested for their antigen binding activities, e.g., by known methods such as ELISA, Western Blot, etc.

2. Activity Assays

In one aspect, assays are provided for identifying anti-PCSK9 antibodies having biological activities. For example, the biological activities of PCSK9 antibodies may include the ability to block, antagonize, inhibit, interfere with, modulate and/or reduce one or more of the biological activities of PCSK9. Antibodies having such biological activity in vivo and/or in vitro are also provided.

In certain embodiments, anti-PCSK9 antibodies bind to human PCSK9 and prevent interaction with the LDLR. In certain embodiments, the invention provides isolated anti-PCSK9 antibodies which specifically bind to PCSK9 and which antagonize the effect on LDLR levels mediated by PCSK9 when measuring the LDLR down regulation in vitro in HepG2 cells disclosed herein.

Exemplary diseases that may be diagnosed with an antibody of the invention include cholesterol related diseases (which include “serum cholesterol related diseases”), including any one or more selected from: hypercholesterolemia, heart disease, metabolic syndrome, diabetes, coronary heart disease, stroke, cardiovascular diseases, Alzheimer's disease and general dyslipidemia, characterized in, for example, an elevated total serum cholesterol level, elevated LDL level, elevated triglycerides level, elevated very low density lipoprotein (VLDL) level, and/or lowered HDL level. In one aspect, the invention provides a method for treating or preventing hypercholesterolemia, and/or at least one symptom selected from dyslipidemia, atherosclerosis, cardiovascular disease (CVD) or coronary heart disease in an individual, comprising administering to the individual an effective amount of an anti-PCSK9 antibody. In certain embodiments, the invention provides an effective amount of an anti-PCSK9 antibody for use in treating or preventing hypercholesterolemia, and/or at least one symptom selected from dyslipidemia, atherosclerosis, CVD or coronary heart disease in a subject. The invention further provides the use of an effective amount of an anti-PCSK9 antibody that antagonizes extracellular or circulating PCSK9 in the manufacture of a medicament for treating or preventing hypercholesterolemia, and/or at least one symptom selected from dyslipidemia, atherosclerosis, CVD or coronary heart disease in an individual.

### EXAMPLES

The examples of methods and compositions in the present invention are as follows. It will be understood that many other examples can be performed according to general descriptions indicated above. In the examples of the present invention, where specific conditions are not described, the experiments are generally conducted under conventional conditions as described in for example, Antibody Technology Laboratory Manual and Molecular Cloning Manual of Cold Spring Harbor, or under conditions proposed by the material or product manufacturers. Where the source of the reagents is not specifically given, the reagents are commercially available conventional reagents.

### Example 1. Preparation of PCSK9 Antigen and Test Protein

UniProt Proprotein convertase subtilisin/kexin type 9 (human PCSK9, Uniprot: Q8MBP7) was used as the template of PCSK9 in the present invention. Optionally, different labels such as his-tag or peptide promoting immunization such as PADRE peptide were fused to PCSK9 protein, then the fusion protein was cloned into the pTT5 vector (Biovector, Cat #: 102762) or the pTargeT vector (promega, A1410), and transiently expressed in 293 cells or stably expressed in CHO-S. Purification steps were performed with conventional methods and the antigen and test protein of the present invention were obtained. The particular sequences are shown in SEQ ID NOs:1-9. The obtained proteins or mutant proteins thereof (such as PCSK9 D374Y mutation, PCSK9-Y) were used as antigens for preparing the anti-human PCSK9 monoclonal antibody and for selecting library.

PCSK9 with His-tag: PCSK9-His6, used as an immunogen for immunizing mice or used as detection reagent.

SEQ ID NO: 1
MGTVSSRRSWWPLPLLLLLLLLLGPAGARAQEDEDGDYEELVLALRS
EEDGLAEAPEHGTTATFHRCAKDPWRLPGTYVVVLKEETHLSQSERT
ARRLQAQAARRGYLTKILHVFHGLLPGFLVKMSGDLLELALKLPHVD
YIEEDSSVFAQSIPWNLERITPPRYRADEYQPPDGGSLVEVYLLDTS
IQSDHREIEGRVMVTDFENVPEEDGTRFHRQASKCDSHGTHLAGVVS
GRDAGVAKGASMRSLRVLNCQGKGTVSGTLIGLEFIRKSQLVQPVGP
LVVLLPLAGGYSRVLNAACQRLARAGVVLVTAAGNFRDDACLYSPAS
APEVITVGATNAQDQPVTLGTLGTNFGRCVDLFAPGEDIIGASSDCS
TCFVSQSGTSQAAAHVAGIAAMMLSAEPELTLAELRQRLIHFSAKDV
INEAWFPEDQRVLTPNLVAALPPSTHGAGWQLFCRTVWSAHSGPTRM
ATAVARCAPDEELLSCSSFSRSGKRRGERMEAQGGKLVCRAHNAFGG
EGVYAIARCCLLPQANCSVHTAPPAEASMGTRVHCHQQGHVLTGCSS
HWEVEDLGTHKPPVLRPRGQPNQCVGHREASIHASCCHAPGLECKVK
EHGIPAPQEQVTVACEEGWTLTGCSALPGTSHVLGAYAVDNTCVVRS
RDVSTTGSTSEGAVTAVAICCRSRHLAQASQELQHHHHHH
NOTE: Underlined sequence is a signal peptide, and italic part is His6-tag sequence.

PCSK9 with PADRE peptide and His-tag: PCSK9-PADRE-His6, used as an immunogen, wherein the contained PADRE peptide can promote immunization;

SEQ ID NO: 2
MGTVSSRRSWWPLPLLLLLLLLLGPAGARAQEDEDGDYEELVLALRSEED
GLAEAPEHGTTATFHRCAKDPWRLPGTYVVVLKEETHLSQSERTARRLQA
QAARRGYLTKILHVFHGLLPGFLVKMSGDLLELALKLPHVDYIEEDSSVF
AQSIPWNLERITPPRYRADEYQPPDGGSLVEVYLLDTSIQSDHREIEGRV
MVTDFENVPEEDGTRFHRQASKCDSHGTHLAGVVSGRDAGVAKGASMRSL
RVLNCQGKGTVSGTLIGLEFIRKSQLVQPVGPLVVLLPLAGGYSRVLNAA
CQRLARAGVVLVTAAGNFRDDACLYSPASAPEVITVGATNAQDQPVTLGT
LGTNFGRCVDLFAPGEDIIGASSDCSTCFVSQSGTSQAAAHVAGIAAMML
SAEPELTLAELRQRLIHFSAKDVINEAWFPEDQRVLTPNLVAALPPSTHG
AGWQLFCRTVWSAHSGPTRMATAVARCAPDEELLSCSSFSRSGKRRGERM
EAQGGKLVCRAHNAFGGEGVYAIARCCLLPQANCSVHTAPPAEASMGTRV
HCHQQGHVLTGCSSHWEVEDLGTHKPPVLRPRGQPNQCVGHREASIHASC
CHAPGLECKVKEHGIPAPQEQVTVACEEGWTLTGCSALPGTSHVLGAYAV

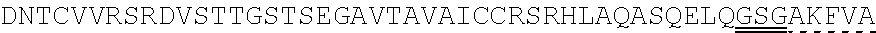

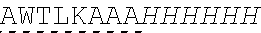

NOTE:
Underlined sequence is a signal peptide, double underlined sequence is a linker, the dashed line sequence is PADRE peptide, and italic part is the His6-tag.

Fusion protein of PCSK9 containing TEV cleavage site and His-tag: PCSK9-TEV-His6, which can be digested with TEV enzyme to obtain N-PCSK9 (N terminal PCSK9 domain), as an immunogen;

SEQ ID NO: 3
MGTVSSRRSWWPLPLLLLLLLLLGPAGARAQEDEDGDYEELVLALR
SEEDGLAEAPEHGTTATFHRCAKDPWRLPGTYVVVLKEETHLSQSE
RTARRLQAQAARRGYLTKILHVFHGLLPGFLVKMSGDLLELALKLP
HVDYIEEDSSVFAQSIPWNLERITPPRYRADEYQPPDGGSLVEVYL
LDTSIQSDHREIEGRVMVTDFENVPEEDGTRFHRQASKCDSHGTHL
AGVVSGRDAGVAKGASMRSLRVLNCQGKGTVSGTLIGLEFIRKSQL
VQPVGPLVVLLPLAGGYSRVLNAACQRLARAGVVLVTAAGNFRDDA
CLYSPASAPEVITVGATNAQDQPVTLGTLGTNFGRCVDLFAPGEDI
IGASSDCSTCFVSQSGTSQAAAHVAGIAAMMLSAEPELTLAELRQR
LIHFSAKDVINEAWFPEDQRVLTPNLVAALPPSTHENLYFQGAGWQ
LFCRTVWSAHSGPTRMATAVARCAPDEELLSCSSFSRSGKRRGERM
EAQGGKLVCRAHNAFGGEGVYAIARCCLLPQANCSVHTAPPAEASM
GTRVHCHQQGHVLTGCSSHWEVEDLGTHKPPVLRPRGQPNQCVGHR
EASIHASCCHAPGLECKVKEHGIPAPQEQVTVACEEGWTLTGCSAL
PGTSHVLGAYAVDNTCVVRSRDVSTTGSTSEGAVTAVAICCRSRHL
AQASQELQHHHHHH
NOTE: Underlined sequence is a signal peptide, double underlined sequence is TEV cleavage site, and italic part is the His6-tag.

PCSK9-D374Y mutant protein with His-tag: PCSK9-D374Y-His6, as a detection reagent;

SEQ ID NO: 4
MGTVSSRRSWWPLPLLLLLLLLLGPAGARAQEDEDGDYEELVLAL
RSEEDGLAEAPEHGTTATFHRCAKDPWRLPGTYVVVLKEETHLSQ
SERTARRLQAQAARRGYLTKILHVFHGLLPGFLVKMSGDLLELAL
KLPHVDYIEEDSSVFAQSIPWNLERITPPRYRADEYQPPDGGSLV
EVYLLDTSIQSDHREIEGRVMVTDFENVPEEDGTRFHRQASKCDS
HGTHLAGVVSGRDAGVAKGASMRSLRVLNCQGKGTVSGTLIGLEF
IRKSQLVQPVGPLVVLLPLAGGYSRVLNAACQRLARAGVVLVTAA
GNFRDDACLYSPASAPEVITVGATNAQDQPVTLGTLGTNFGRCVD
LFAPGEDIIGASSYCSTCFVSQSGTSQAAAHVAGIAAMMLSAEPE
LTLAELRQRLIHFSAKDVINEAWFPEDQRVLTPNLVAALPPSTHG
AGWQLFCRTVWSAHSGPTRMATAVARCAPDEELLSCSSFSRSGKR
RGERMEAQGGKLVCRAHNAFGGEGVYAIARCCLLPQANCSVHTAP
PAEASMGTRVHCHQQGHVLTGCSSHWEVEDLGTHKPPVLRPRGQP
NQCVGHREASIHASCCHAPGLECKVKEHGIPAPQEQVTVACEEGW
TLTGCSALPGTSHVLGAYAVDNTCVVRSRDVSTTGSTSEGAVTAV
AICCRSRHLAQASQELQHHHHHH
NOTE: Underlined sequence is a signal peptide, and italic part is the His6-tag.

PCSK9 protein inserted with biotin receiving peptide BP15 and His-tag: PCSK9-BP15-His6, as a detection reagent, biotin can be labeled to the BP15 peptide position during the expression, avoiding the biotin labeling in vitro and consequently avoiding possible conformational changes.

SEQ ID NO: 5
MGTVSSRRSWWPLPLLLLLLLLLGPAGARAQEDEDGDYEELVLAL
RSEEDGLAEAPEHGTTATFHRCAKDPWRLPGTYVVVLKEETHLSQ
SERTARRLQAQAARRGYLTKILHVFHGLLPGFLVKMSGDLLELAL
KLPHVDYIEEDSSVFAQSIPWNLERITPPRYRADEYQPPDGGSLV
EVYLLDTSIQSDHREIEGRVMVTDFENVPEEDGTRFHRQASKCDS
HGTHLAGVVSGRDAGVAKGASMRSLRVLNCQGKGTVSGTLIGLEF
IRKSQLVQPVGPLVVLLPLAGGYSRVLNAACQRLARAGVVLVTAA
GNFRDDACLYSPASAPEVITVGATNAQDQPVTLGTLGTNFGRCVD
LFAPGEDIIGASSDCSTCFVSQSGTSQAAAHVAGIAAMMLSAEPE
LTLAELRQRLIHFSAKDVINEAWFPEDQRVLTPNLVAALPPSTHG
AGWQLFCRTVWSAHSGPTRMATAVARCAPDEELLSCSSFSRSGKR
RGERMEAQGGKLVCRAHNAFGGEGVYAIARCCLLPQANCSVHTAP
PAEASMGTRVHCHQQGHVLTGCSSHWEVEDLGTHKPPVLRPRGQP
NQCVGHREASIHASCCHAPGLECKVKEHGIPAPQEQVTVACEEGW
TLTGCSALPGTSHVLGAYAVDNTCVVRSRDVSTTGSTSEGAVTAV
AICCRSRHLAQASQELQGSTSGSGLNDIFEAQKIEWHEHHHHHH
NOTE: Underlined sequence is a signal peptide,double underlined sequence is the biotin receiving peptide, and italic part is the His6-tag.

PCSK9 D374Y mutant protein inserted with biotin receiving peptide BP15 and His-tag: PCSK9-D374Y-BP15-His6, as a detection protein:

SEQ ID NO: 6
MGTVSSRRSWWPLPLLLLLLLLLGPAGARAQEDEDGDYEELVLAL
RSEEDGLAEAPEHGTTATFHRCAKDPWRLPGTYVVVLKEETHLSQ
SERTARRLQAQAARRGYLTKILHVFHGLLPGFLVKMSGDLLELAL
KLPHVDYIEEDSSVFAQSIPWNLERITPPRYRADEYQPPDGGSLV
EVYLLDTSIQSDHREIEGRVMVTDFENVPEEDGTRFHRQASKCDS
HGTHLAGVVSGRDAGVAKGASMRSLRVLNCQGKGTVSGTLIGLEF
IRKSQLVQPVGPLVVLLPLAGGYSRVLNAACQRLARAGVVLVTAA
GNFRDDACLYSPASAPEVITVGATNAQDQPVTLGTLGTNFGRCVD
LFAPGEDIIGASSYCSTCFVSQSGTSQAAAHVAGIAAMMLSAEPE
LTLAELRQRLIHFSAKDVINEAWFPEDQRVLTPNLVAALPPSTHG
AGWQLFCRTVWSAHSGPTRMATAVARCAPDEELLSCSSFSRSGKR
RGERMEAQGGKLVCRAHNAFGGEGVYAIARCCLLPQANCSVHTAP
PAEASMGTRVHCHQQGHVLTGCSSHWEVEDLGTHKPPVLRPRGQP
NQCVGHREASIHASCCHAPGLECKVKEHGIPAPQEQVTVACEEGW
TLTGCSALPGTSHVLGAYAVDNTCVVRSRDVSTTGSTSEGAVTAV
AICCRSRHLAQASQELQGSTSGSGLNDIFEAQKIEWHEHHHHHH
NOTE: Underlined sequence is a signal peptide, double underlined sequence is the biotin receiving peptide, and italic part is the His6-tag.

PCSK9 receptor protein LDLR extracellular domain with Flag tag and His-tag: LDLR-ECD-Flag-His6 as a detection reagent;

SEQ ID NO: 7
MGPWGWKLRWTVALLLAAAGTAVGDRCERNEFQCQDGKCISYKWVCDGS
AECQDGSDESQETCLSVTCKSGDFSCGGRVNRCIPQFWRCDGQVDCDNG
SDEQGCPPKTCSQDEFRCHDGKCISRQFVCDSDRDCLDGSDEASCPVLT
CGPASFQCNSSTCIPQLWACDNDPDCEDGSDEWPQRCRGLYVFQGDSSP
CSAFEFHCLSGECIHSSWRCDGGPDCKDKSDEENCAVATCRPDEFQCSD
GNCIHGSRQCDREYDCKDMSDEVGCVNVTLCEGPNKFKCHSGECITLDK
VCNMARDCRDWSDEPIKECGTNECLDNNGGCSHVCNDLKIGYECLCPDG
FQLVAQRRCEDIDECQDPDTCSQLCVNLEGGYKCQCEEGFQLDPHTKAC
KAVGSIAYLFFTNRHEVRKMTLDRSEYTSLIPNLRNVVALDTEVASNRI
YWSDLSQRMICSTQLDRAHGVSSYDTVISRDIQAPDGLAVDWIHSNIYW
TDSVLGTVSVADTKGVKRKTLFRENGSKPRAIVVDPVHGFMYWTDWGTP
AKIKKGGLNGVDIYSLVTENIQWPNGITLDLLSGRLYWVDSKLHSISSI
DVNGGNRKTILEDEKRLAHPFSLAVFEDKVFWTDIINEAIFSANRLTGS
DVNLLAENLLSPEDMVLFHNLTQPRGVNWCERTTLSNGGCQYLCLPAPQ
INPHSPKFTCACPDGMLLARDMRSCLTEAEAAVATQETSTVRLKVSSTA
VRTQHTTTRPVPDTSRLPGATPGLTTVEIVTMSHQALGDVAGRGNEKKP
SSVRDYKDDDDKHHHHHH
NOTE: Underlined sequence is a signal peptide, double underlined sequence is the Flag tag, and italic part is the His6-tag.

Fusion protein of truncated LDLR extracellular domain and hIgG1 Fc (with PCSK9 binding activity): LDLR-sECD-Fc (hIgG1), as a detection reagent;

SEQ ID NO: 8
MEFGLSWLFLVAILKGVQCGTNECLDNNGGCSHVCNDLKIGYECLCPDG
FQLVAQRRCEDIDECQDPDTCSQLCVNLEGGYKCQCEEGFQLDPHTKAC
KEPKSSDKTHTCPPCPAPELLGGPSVFLFPPKPKDTLMISRTPEVTCVV
VDVSHEDPEVKFNWYVDGVEVHNAKTKPREEQYNSTYRVVSVLTVLHQD
WLNGKEYKCKVSNKALPAPIEKTISKAKGQPREPQVYTLPPSRDELTKN
QVSLTCLVKGFYPSDIAVEWESNGQPENNYKTTPPVLDSDGSFFLYSKL
TVDKSRWQQGNVFSCSVMHEALHNHYTQKSLSLSPGK
NOTE: Underlined sequence is a signal peptide, double underlined sequence is the truncated LDLR extracellular domain with PCSK9 binding activity (LDLR-sECD), and italic part is the hIgGl-Fc.

Fusion protein of further truncated LDLR extracellular domain and hIgG1 Fc (with PCSK9 binding activity): LDLR-ssECD-Fc (hIgG1), as a detection reagent;

SEQ ID NO: 9
MEFGLSWLFLVAILKGVQCGTNECLDNNGGCSHVCNDLKIGYECLCPDG
FQLVAQRRCEDIDEPKSSDKTHTCPPCPAPELLGGPSVFLFPPKPKDTL
MISRTPEVTCVVVDVSHEDPEVKFNWYVDGVEVHNAKTKPREEQYNSTY
RVVSVLTVLHQDWLNGKEYKCKVSNKALPAPIEKTISKAKGQPREPQVY
TLPPSRDELTKNQVSLTCLVKGFYPSDIAVEWESNGQPENNYKTTPPVL
DSDGSFFLYSKLTVDKSRWQQGNVFSCSVMHEALHNHYTQKSLSLSPGK
NOTE: Underlined sequence is a signal peptide, double underlined sequence is the further truncated LDLR extracellular domain with PCSK9 binding activity (LDLR-ssECD), and italic part is the hIgGl-Fc.

### Example 2. Preparation of Anti-human PCSK9 Monoclonal Antibody

The anti-human PCSK9 monoclonal antibody was produced by immunizing mice. Experimental SJL white mice, female, 6 weeks old (Beijing Weitong Lihua Experimental Animal Technology Co., Ltd., animal production license number: SCXK (Beijing) 2012-0001).

Feeding environment: SPF level. After the mice were purchased, the animals were kept in the laboratory for 1 week, 12/12 hours light/dark cycle, temperature 20-25° C., humidity 40-60%. The mice that adapted to the environment were immunized according to the following two schemes (A/B), each group of 6-10 mice. Immunized antigen was human PCSK9 with His tag: PCSK9-His6 (SEQ ID NO: 1), PCSK9-PADRE-His6 (SEQ ID NO: 2) and N-PCSK9 (SEQ ID NO: 3).

Scheme A: emulsifying with Freund's adjuvant (sigma Lot Num: F5881/F5506): first immunization with Complete Freund's adjuvant (CFA), booster immunization with Incomplete Freund's adjuvant (IFA). The ratio of antigen and adjuvant was 1:1, 100 μg/mouse (first immunization), 50 μg/mouse (booster immunization). On day 0, mice were intraperitoneally (IP) injected with 100 μg/mouse of emulsified antigens, after first immunization, once every two weeks, total 6-8 weeks.

Scheme B: Cross immunization with Titermax (sigma Lot Num: T2684) and Alum (Thremo Lot Num: 77161). The ratio of antigen and adjuvant (titermax) was 1:1, and the ratio of antigen and adjuvant (Alum) was 3:1, 10-20 μg/mouse (first immunization), 5 μg/mouse (booster immunization). On day 0, mice were intraperitoneally (IP) injected with 20/10 μg/mouse emulsified antigen, once a week after first immunization, Titermax and Alum were alternately used, total 6-11 weeks. Four weeks after immunization, back injection or intraperitoneal injection with antigen was selected according to the swelling conditions on back and abdomen.

### Example 3. Library Construction

Spleens of the immunized mice were sieved via filter, washed with PBS. Then the RNA was extracted and cDNA was obtained by reverse transcription. The antibody residues were numbered according to Kabat (Kabat et al., Sequences of proteins of immunological interest, 5th Ed., Public Health Service, National Institutes of Health, Bethesda, Md. (1991)). The heavy and light chains were amplified separately using upstream primer mixture and downstream primer mixture. Primers were designed based on Brocks et al. 2001. SfiI restriction site and protective bases were incorporated into the heavy chain upstream primer, and partial sequence of the linker was incorporated into the downstream primer; partial sequence of the linker, which was complementary to the heavy chain downstream primer, was incorporated into the light chain upstream primer, another sfiI restriction site and protected bases were incorporated into the downstream primer. The amplified VH and VL fragments were recovered via gel recovery, and spliced into scFv (comprising VH-(G4S) 3-VL) by over-lap PCR. The phagemid vector and scFv were both digested with sfiI, and then connected with each other. E. coli strain SS320 was electro-transformed and the capacity of the library was approximately 1E9.

### Example 4. Screening

Panning was performed by liquid-phase method after the E. coli library was packaged into phage particles with helper phage (NEB, N0315S): Phages were bound to biotinylated PCSK9 in liquid phase and were separated by streptavidin beads. After two rounds of panning, monoclonal antibodies were picked from the phage for phage ELISA assay. The assay was divided into two parts: binding activity and blocking activity. Binding activity: ELISA plate was coated with 2 ng/μl streptavidin, and incubated with the 1 ng/μl biotinylated PCSK9 (SEQ ID NO: 5), then phage supernatant diluted with 1:1 blocking buffer (1×PBS+2% skim milk) was added and finally detected with anti-M13 HRP (GE, 27-9421-01); blocking activity: similar to binding activity, except that 50 ng/μl final concentration of LDLR-Fc (SEQ ID NO: 8) was added during the incubation of phage supernatant. The ELISA OD45 value obtained from the binding activity test was divided by the ELISA OD45 value obtained from the blocking activity test, and then clones with resulting value greater than 2.0 were screened, including murine clone mAb-011, for sequencing, and further screening.

### Example 5. Expression and Identification of Chimeric Antibodies

The selected clones (including mAb-011) were constructed into an IgG1/κ chimeric antibody expression vector and transiently expressed in mammalian cells. After Protein A affinity purification, the binding activity of PCSK9 (WT PCSK9, SEQ ID NO: 5) and PCSK9-Y (mutant PCSK9, SEQ ID NO: 6) were tested (test examples 1 and 2), then the EC50 values were calculated; and blocking activity (test examples 3 and 4) of wild-type PCSK9 and PCSK9-Y were also tested, and their IC50 values were calculated. The binding activity was tested by using streptavidin-coated plates and incubating biotinylated PCSK9 (or PCSK9-Y) and then incubating serially diluted chimeric antibodies. The blocking activity was tested by using LDLR-Fc-coated plates, blocking the plates, and incubating serially diluted chimeric antibodies and biotinylated PCSK9 (or biotinylated PCSK9-Y) at the same time, and then incubating streptavidin HRP for detection. The chimeric antibody ch-011 with better activity (see Table 2) was screened as a key molecule for subsequent humanization.

TABLE 2
Binding and blocking activity of the chimeric antibody:

Ch-011

| | Binding activity | Blocking activity |
|---|---|---|
| Receptor | EC50(ug/ml) | IC50(ug/ml) |
| PCSK9 WT | 0.005 | 0.263 |
| PCSK9Y mutant | 0.050 | 3.113 |

The results of the binding test, in which the PCSK9 chimeric antibody ch-011 screened in the present invention was tested for binding activity to the PCSK9/PCSK9-Y protein, showed that: ch-011 antibody has an effective binding activity to PCSK9/PCSK9-Y, and has a higher binding activity to PCSK9.

The results of the blocking test, in which the PCSK9 chimeric antibody ch-011 screened in the present invention was tested for activity of blocking the binding of LDLR with PCSK9/PCSK9-Y, showed that: ch-011 antibody has an effective activity of blocking the binding of LDLR with PCSK9/PCSK9-Y, and has a higher blocking effect on the binding of LDLR with PCSK9.

### Example 6. Humanization and Identification of mAb-011

The murine-derived clone mAb-011 was chosen for humanization based on the results of the chimeric antibody experiment. Humanization strategy was the CDR-graft strategy. After aligning with the human germline gene database of heavy light chain variable regions, the germline gene with the highest homology to murine mAb-011 sequence was selected as a template. The humanized light chain templates for the murine antibody mAb-011 are IGKV1-39*01 and hjk4.1, and the humanized heavy chain templates are IGHV1-2*02 and hjh6.1, and the humanized variable region sequences are as follows:

SEQ ID NO: 10
>h011-1 VH (CDR graft)
QVQLVQSGAEVKKPGASVKVSCKASGYTFTGYTIHWVRQAPG
QGLEWMGYINPSSTYTKFNQKFKDRVTMTRDTSISTAYMELS
RLRSDDTAVYYCARERIYSNYWFFDVWGQGTTVTVSS
SEQ ID NO: 11
>h011-1 VL (CDR graft)
DIQMTQSPSSLSASVGDRVTITCKASQNVYTAVAWYQQKPGK
APKLLIYSASNRYTGVPSRFSGSGSGTDFTLTISSLQPEDFA
TYYCQQYSSYPYTFGGGTKVEIK

Different back mutations were selected for combination (Table 3) after CDR grafting. The designed humanized sequences were fully-gene synthesized and heavy and light chains were combined with each other for mammalian expression (Table 4). After purification of the protein, the blocking activity test was performed similarly to that for chimeric antibodies (see test 3 and 4 for the method and the results are shown in Table 5). Some cloned proteins were selected and were determined for dissociation constants by Surface Plasmon resonance (SPR) (Biacore X100, GE, see test 5). The sample to be tested was captured by the Amine-Coupling Anti-Fc pAb (GE) on the CM5 chip (GE). PCSK9 (SEQ ID NO: 1) or PCSK9-Y (SEQ ID NO: 4) was the mobile phase, working buffer was 1×HBS-EP⁺, pH 7.4, regeneration bu0ffer was 3 M MgCl₂. The results are shown in Table 6.

TABLE 3
Template selection and back mutation design for mAb-011

| mAb-011_VH | | mAb-011_VL | |
|---|---|---|---|
| h011_VH.1 | Graft | h011_VL.1 | Graft |
| h011_VH.1A | R72A, T74K | h011_VL.1A | Q3V |
| h011_VH.1B | R72A, T74K, M48V, V68A | h011_VL.1B | Q3V, A43S, Y87F |
| h011_VH.1C | R72A, T74K, V68A, M70L | | |
| h011_VH.1D | R72A, T74K, V68A, M70L, M48V | | |
| h011_VH.1E | R72A, T74K, V68A, M70L, M48V, G49A | | |
| h011_VH.1F | R72A, T74K, V68A, M70L, M48V, G49A, R67K, R38K | | |

TABLE 4
Combination of heavy and light chains

| | h011_VL.1 | h011_VL.1A | h011_VL.1B |
|---|---|---|---|
| h011_VH.1 | h011-1 | h011-2 | h011-3 |
| h011_VH.1A | h011-4 | h011-5 | h0117-6 |
| h011_VH.1B | h011-7 | h011-8 | h011-9 |
| h011_VH.1C | h011-10 | h011-11 | h011-12 |
| h011_VH.1D | h011-13 | h011-14 | h011-15 |
| h011_VH.1E | h011-16 | h011-17 | h011-18 |
| h011_VH.1F | h011-19 | h011-20 | h011-21 |

Note: The table indicated the sequence obtained by combining various mutations. As indicated by h011-5, there were two mutations (heavy chain h011_VH.1A and light chain h011_VL.1A) on the humanized antibody h011-5. And so on.

An ELISA test was performed to detect the binding to PCSK9 or PCSK9-Y (see Test 1 and 2). The positive cells detected in the ELISA test were further used in an ELISA test to detect blocking of the binding of PCSK9/PCSK9-Y to LDLR (see test 3 and 4). The light and heavy chain combination was finally determined as h011-VH.1 and h011-VL1B. The heavy and light chain variable region sequences of the humanized h011-3 were as follows:

SEQ ID NO: 12
>h011-3 VH
QVQLVQSGAEVKKPGASVKVSCKASGYTFTGYTIHWVRQAPGQ
GLEWMGYINPSSTYTKFNQKFKDRVTMTRDTSISTAYMELSRL
RSDDTAVYYCARERIYSNYWFFDVWGQGTTVTVSS
SEQ ID NO: 13
>h011-3 VL
DIVMTQSPSSLSASVGDRVTITCKASQNVYTAVAWYQQKPGKS
PKLLIYSASNRYTGVPSRFSGSGSGTDFTLTISSLQPEDFATY
FCQQYSSYPYTFGGGTKVEIK

TABLE 5
Blocking activities of chimeric and humanized antibodies
on the binding of LDLR-FC to PCSK9 or PCSK9-Y

IC50

| Clone No. | PCSK9 (ug/ml) | PCSK9-Y(ug/ml) |
|---|---|---|
| ch-011 | 0.2028 | 3.552 |
| h011-3 | 0.2055 | 5.127 |

The results of the binding test of the PCSK9 chimeric or humanized antibodies screened by the present invention to wild type/mutant PCSK9 protein showed that: h011-3 and ch-011 antibodies have high binding activity to wild type/mutant PCSK9, and the binding activity of h011-3 and ch-011 antibodies to wild type PCSK9 is higher.

TABLE 6
Dissociation constants of some samples after humanization

| Analytical | | | | |
|---|---|---|---|---|
| substrate | sample | ka (1/Ms) | kd (1/s) | KD (M) |
| PCSK9 | ch-011 | 1.12E+05 | 4.32E−05 | 3.85E−10 |
| | h011-3 | 7.13E+04 | 1.88E−05 | 2.63E−10 |
| PCSK9-Y | ch-011 | 2.43E+05 | 3.27E−02 | 1.35E−07 |
| | h011-3 | 1.68E+05 | 5.78E−03 | 3.45E−08 |

Note:
ch011: mAb-011chimeric antibody

Biacore test results of the PCSK9 chimeric or humanized antibodies screened by the present invention to wild type/mutant PCSK9 showed that: h011-3 and ch-011 have lower equilibrium dissociation constants, and high affinity. h011-3 and ch-011 have higher affinity to wild-type PCSK9.

### Example 7. Affinity Maturation of h011-3

We decided to carry out the affinity maturation against PCSK9-Y with h011-3, as the affinity of murine mAb-011 and its humanized antibody h011-3 to PCSK9-Y is low. M13 phage display technology was used in the affinity maturation. Codon-based primers (during the synthesis of primers, single codon consists of wild-type codon and NNK) were adopted to introduce mutations in each CDR, and a separate phage display library was constructed for each CDR. Based on the length of the CDRs, the ratio of NNK and the library size required for the library were adjusted (Table 7).

TABLE 7
Library size and NNK incorporation ratio

| Lib | CDR length | NNK ratio | Lib size |
|---|---|---|---|
| H1 | 5 | 50% | >2E7 |
| H2 | 17 | 20% | >1E8 |
| H3 | 12 | 30% | >1E8 |
| L1 | 11 | 30% | >1E8 |
| L2 | 7 | 50% | >2E7 |
| L3 | 9 | 40% | >1E8 |

The constructed 6 libraries were packaged into phages for panning: associated with biotinylated PCSK9-Y (SEQ ID NO: 6) in liquid phase, captured by streptavidin, elutriated, eluted, then re-infected with E. coli for the next round of panning. The concentration of biotinylated PCSK9-Y was reduced by 10-fold in each round of panning. After 3-4 rounds of panning, a single clone was picked from each library for sequencing verification. According to the enrichment degree of amino acid residues in CDR regions, some clones were selected to construct full-length Ig for expression in mammalian cells; meanwhile the mutations with different enrichment degree of CDRs were combined artificially and constructed as a full-length Ig for mammalian cell expression (as shown in Table 8-11).

After purification, the cloned protein was used in an ELISA to detect the binding to PCSK9 (test 1) and to PCSK9-Y (test 2); then the positive cells detected in the above ELISA were used in an ELISA test to detect blocking of PCSK9-Y/LDLR binding (test 3), and to detect blocking of PCSK9/LDLR binding (test 4). The results are shown in Tables 12-15.

The results indicated that the PCSK9 antibodies obtained by the present invention have high binding activity to PCSK9 and PCSK9-Y, and can effectively block the binding of PCSK9/PCSK9-Y to LDLR.

TABLE 8
CDR sequences of enriched clones and artificial combinatorial clones

| Clone No. | HCDR1 | HCDR2 | HCDR3 | LCDR1 | LCDR2 | LCDR3 |
|---|---|---|---|---|---|---|
| h011-3 | GYTIH | YINPSSTYTKFNQKFKD | ARERIYSNYWFFDV | KASQNVYTAVA | SASNRYT | QQYSSYPYT |
| SEQ ID NO | 14 | 15 | 16 | 17 | 18 | 19 |
| h011-3050 | ----- | e-l-------------- | -------------- | ----------- | ------- | --------- |
| SEQ ID NO | 14 | 22 | 16 | 17 | 18 | 19 |
| h011-3058 | ----- | e----g----------- | -------------- | ----------- | ------- | --------- |
| SEQ ID NO | 14 | 23 | 16 | 17 | 18 | 19 |
| h011-3065 | --d-- | ----------------- | -------------- | ----------- | ------- | --------- |
| SEQ ID NO | 20 | 15 | 16 | 17 | 18 | 19 |
| h011-3070 | ----- | ----------------- | ---n--f------r | ----------- | ------- | --------- |
| SEQ ID NO | 14 | 15 | 28 | 17 | 18 | 19 |
| h011-3073 | ----- | ----------------- | ---n-f-------r | ----------- | ------- | --------- |
| SEQ ID NO | 14 | 15 | 29 | 17 | 18 | 19 |
| h011-3093 | ----- | ----------------- | -------------- | ----------- | emv---- | --------- |
| SEQ ID NO | 14 | 15 | 16 | 17 | 35 | 19 |
| h011-3095 | ----- | ----------------- | -------------- | ----------- | e------ | --------- |
| SEQ ID NO | 14 | 15 | 16 | 17 | 36 | 19 |
| h011-3111 | ----- | ----------------- | -------------- | -------we-d | ------- | --------- |
| SEQ ID NO | 14 | 15 | 16 | 31 | 18 | 19 |
| h011-3118 | ----- | ----------------- | -------------- | -------we-v | ------- | --------- |
| SEQ ID NO | 14 | 15 | 16 | 32 | 18 | 19 |
| h011-3120 | ----- | ----------------- | -------------- | ----------- | ------- | --f-wf--- |
| SEQ ID NO | 14 | 15 | 16 | 17 | 18 | 38 |
| h011-3121 | ----- | ----------------- | -------------- | ----------- | ------- | --l--q-e- |
| SEQ ID NO | 14 | 15 | 16 | 17 | 18 | 39 |
| h011-3133 | --e-- | -----a----------- | -------------- | ----------- | ------- | --------- |
| SEQ ID NO | 21 | 24 | 16 | 17 | 18 | 19 |
| h011-3147 | ----- | e-i-------------- | -------------- | ----------- | ------- | --------- |
| SEQ ID NO | 14 | 25 | 16 | 17 | 18 | 19 |
| h011-3174 | ----- | ----------------- | -----f-------- | ----------- | q------ | --------- |
| SEQ ID NO | 14 | 15 | 30 | 17 | 37 | 19 |
| h011-3181 | ----- | ----------------- | -------------- | ----------- | ------- | --l----d- |
| SEQ ID NO | 14 | 15 | 16 | 17 | 18 | 40 |
| h011-3187 | ----- | ----------------- | -------------- | ----------- | ------- | --l--s-e- |
| SEQ ID NO | 14 | 15 | 16 | 17 | 18 | 41 |
| h011-3190 | ----- | e---------------- | -------------- | ----------- | ------- | --------- |
| SEQ ID NO | 14 | 26 | 16 | 17 | 18 | 19 |
| h011-3191 | ----- | --v-------------- | -------------- | ----------- | ------- | --------- |
| SEQ ID NO | 14 | 27 | 16 | 17 | 18 | 19 |
| h011-3192 | ----- | ----------------- | -------------- | --------e-- | ------- | --------- |
| SEQ ID NO | 14 | 15 | 16 | 33 | 18 | 19 |
| h011-3193 | ----- | ----------------- | -------------- | ----------d | ------- | --------- |
| SEQ ID NO | 14 | 15 | 16 | 34 | 18 | 19 |
| h011-3194 | ----- | ----------------- | -------------- | ----------- | ------- | --l------ |
| SEQ ID NO | 14 | 15 | 16 | 17 | 18 | 42 |
| h011-3195 | ----- | ----------------- | -----f-------- | ----------- | ------- | --------- |
| SEQ ID NO | 14 | 15 | 30 | 17 | 18 | 19 |

The CDR sequences of the artificial combination clones in Table 8 above could be defined and summarized in Table 9:

TABLE 9
CDR sequences of the antibodies
in the present invention

| Clone | | | | | | |
|---|---|---|---|---|---|---|
| No. | HCDR1 | HCDR2 | HCDR3 | LCDR1 | LCDR2 | LCDR3 |
| h011 | GYX¹IH | X²IX³PSX⁴TYTK | AREX⁵IX⁶X⁷ | KASQNVY | X₄X₅X₆ | QQX₇SX₈ |
| 3 | | FNQKFKD | NYWFFDX⁸ | X₁X₂VX₃ | NRYT | X₉PX₁₀T |
| SEQ | 43 | 44 | 45 | 46 | 47 | 48 |
| ID NO | | | | | | |

X¹ is selected from T, D or E;
X² is selected from Y or E; X³is selected from N, L, I or V; X⁴ is selected from S, G or A;
X⁵ is selected from R or N; X⁶ is selected from Y or F; X⁷is selected from S or F; X⁸ is selected from V or R;
X₁ is selected from T or W; X₂ is selected from A or E; X₃ is selected from A, D or V;
X₄ is selected from S, E or Q; X₅ is selected from A or M; X₆ is selected from S or V;
X₇ is selected from Y, F or L; X₈ is selected from S or W; X₉ is selected from Y, F, Q or S; X₁₀ is selected from Y, D or E.
The artificial combinations of the light and heavy chain variable region sequences of monoclonal antibodies are shown in Table 10 below:

TABLE 10
Combinations of variable region sequences of monoclonal antibody

| | VH | VL |
|---|---|---|
| Clone No. | SEQ ID NO | SEQ ID NO |
| h011-3 | 12 | 13 |
| h011-3050 | 49 | 13 |
| h011-3058 | 50 | 13 |
| h011-3065 | 51 | 13 |
| h011-3070 | 52 | 13 |
| h011-3073 | 53 | 13 |
| h011-3093 | 12 | 59 |
| h011-3095 | 12 | 60 |
| h011-3111 | 12 | 61 |
| h011-3118 | 12 | 62 |
| h011-3120 | 12 | 63 |
| h011-3121 | 12 | 64 |
| h011-3133 | 54 | 13 |
| h011-3147 | 55 | 13 |
| h011-3174 | 56 | 65 |
| h011-3181 | 12 | 66 |
| h011-3187 | 12 | 67 |
| h011-3190 | 57 | 13 |
| h011-3191 | 58 | 13 |
| h011-3192 | 12 | 68 |
| h011-3193 | 12 | 69 |
| h011-3194 | 12 | 70 |
| h011-3195 | 56 | 13 |

The variable region sequences of the light and heavy chains are shown in Table 11:

| SEQ ID | |
|---|---|
| NO | sequence |
| 50 | QVQLVQSGAEVKKPGASVKVSCKASGYTFTGYTIHWVRQAPGQ |
| | GLEWMGEINPSGTYTKFNQKFKDRVTMTRDTSISTAYMELSRL |
| | RSDDTAVYYCARERIYSNYWFFDVWGQGTTVTVSS |
| 51 | QVQLVQSGAEVKKPGASVKVSCKASGYTFTGYDIHWVRQAPGQ |
| | GLEWMGYINPSSTYTKFNQKFKDRVTMTRDTSISTAYMELSRL |
| | RSDDTAVYYCARERIYSNYWFFDVWGQGTTVTVSS |
| 52 | QVQLVQSGAEVKKPGASVKVSCKASGYTFTGYTIHWVRQAPGQ |
| | GLEWMGYINPSSTYTKFNQKFKDRVTMTRDTSISTAYMELSRL |
| | RSDDTAVYYCARENIYFNYWFFDRWGQGTTVTVSS |
| 53 | QVQLVQSGAEVKKPGASVKVSCKASGYTFTGYTIHWVRQAPGQ |
| | GLEWMGYINPSSTYTKFNQKFKDRVTMTRDTSISTAYMELSRL |
| | RSDDTAVYYCARENIFSNYWFFDRWGQGTTVTVSS |
| 54 | QVQLVQSGAEVKKPGASVKVSCKASGYTFTGYEIHWVRQAPGQ |
| | GLEWMGYINPSATYTKFNQKFKDRVTMTRDTSISTAYMELSRL |
| | RSDDTAVYYCARERIYSNYWFFDVWGQGTTVTVSS |
| 55 | QVQLVQSGAEVKKPGASVKVSCKASGYTFTGYTIHWVRQAPGQ |
| | GLEWMGEIIPSSTYTKFNQKFKDRVTMTRDTSISTAYMELSRL |
| | RSDDTAVYYCARERIYSNYWFFDVWGQGTTVTVSS |
| 56 | QVQLVQSGAEVKKPGASVKVSCKASGYTFTGYTIHWVRQAPGQ |
| | GLEWMGYINPSSTYTKFNQKFKDRVTMTRDTSISTAYMELSRL |
| | RSDDTAVYYCARERIFSNYWFFDVWGQGTTVTVSS |
| 57 | QVQLVQSGAEVKKPGASVKVSCKASGYTFTGYTIHWVRQAPGQ |
| | GLEWMGEINPSSTYTKFNQKFKDRVTMTRDTSISTAYMELSRL |
| | RSDDTAVYYCARERIYSNYWFFDVWGQGTTVTVSS |
| 58 | QVQLVQSGAEVKKPGASVKVSCKASGYTFTGYTIHWVRQAPGQ |
| | GLEWMGYIVPSSTYTKFNQKFKDRVTMTRDTSISTAYMELSRL |
| | RSDDTAVYYCARERIYSNYWFFDVWGQGTTVTVSS |
| 59 | DIVMTQSPSSLSASVGDRVTITCKASQNVYTAVAWYQQKPGKS |
| | PKLLIYEMVNRYTGVPSRFSGSGSGTDFTLTISSLQPEDFATY |
| | FCQQYSSYPYTFGGGTKVEIK |
| 60 | DIVMTQSPSSLSASVGDRVTITCKASQNVYTAVAWYQQKPGKS |
| | PKLLIYEASNRYTGVPSRFSGSGSGTDFTLTISSLQPEDFATY |
| | FCQQYSSYPYTFGGGTKVEIK |
| 61 | DIVMTQSPSSLSASVGDRVTITCKASQNVYWEVDWYQQKPGKS |
| | PKLLIYSASNRYTGVPSRFSGSGSGTDFTLTISSLQPEDFATY |
| | FCQQYSSYPYTFGGGTKVEIK |
| 62 | DIVMTQSPSSLSASVGDRVTITCKASQNVYWEVVWYQQKPGKS |
| | PKLLIYSASNRYTGVPSRFSGSGSGTDFTLTISSLQPEDFATY |
| | FCQQYSSYPYTFGGGTKVEIK |
| 63 | DIVMTQSPSSLSASVGDRVTITCKASQNVYTAVAWYQQKPGKS |
| | PKLLIYSASNRYTGVPSRFSGSGSGTDFTLTISSLQPEDFATY |
| | FCQQFSWFPYTFGGGTKVEIK |
| 64 | DIVMTQSPSSLSASVGDRVTITCKASQNVYTAVAWYQQKPGKS |
| | PKLLIYSASNRYTGVPSRFSGSGSGTDFTLTISSLQPEDFATY |
| | FCQQLSSQPETFGGGTKVEIK |
| 65 | DIVMTQSPSSLSASVGDRVTITCKASQNVYTAVAWYQQKPGKS |
| | PKLLIYQASNRYTGVPSRFSGSGSGTDFTLTISSLQPEDFATY |
| | FCQQYSSYPYTFGGGTKVEIK |
| 66 | DIVMTQSPSSLSASVGDRVTITCKASQNVYTAVAWYQQKPGKS |
| | PKLLIYSASNRYTGVPSRFSGSGSGTDFTLTISSLQPEDFATY |
| | FCQQLSSYPDTFGGGTKVEIK |
| 67 | DIVMTQSPSSLSASVGDRVTITCKASQNVYTAVAWYQQKPGKS |
| | PKLLIYSASNRYTGVPSRFSGSGSGTDFTLTISSLQPEDFATY |
| | FCQQLSSSPETFGGGTKVEIK |
| 68 | DIVMTQSPSSLSASVGDRVTITCKASQNVYTEVAWYQQKPGKS |
| | PKLLIYSASNRYTGVPSRFSGSGSGTDFTLTISSLQPEDFATY |
| | FCQQYSSYPYTFGGGTKVEIK |
| 69 | DIVMTQSPSSLSASVGDRVTITCKASQNVYTAVDWYQQKPGKS |
| | PKLLIYSASNRYTGVPSRFSGSGSGTDFTLTISSLQPEDFATY |
| | FCQQYSSYPYTFGGGTKVEIK |
| 70 | DIVMTQSPSSLSASVGDRVTITCKASQNVYTAVAWYQQKPGKS |
| | PKLLIYSASNRYTGVPSRFSGSGSGTDFTLTISSLQPEDFATY |
| | FCQQLSSYPYTFGGGTKVEIK |

### Example 8. Construction and Expression of IgG1 and IgG1-YTE Formats of Anti-Human PCSK9 Humanized Antibodies

PCR primers were designed to construct VH/VK gene fragments of humanized antibodies, the VH/VK gene fragments were then homologously recombined with expression vector pHr (with signal peptide and constant region gene (CH1-FC/CL) fragment) to construct full-length antibody expression vector VH-CH1-FC-pHr/VK-CL-pHr. The IgG1-YTE antibody format can be obtained via point mutation of the IgG1 antibody format. Several antibody formats were designed and constructed, 1) h011-3133-WT: h011-3133-IgG1 format, i.e., humanized sequence combination h011-3133, in combination with the heavy chain constant regions from human IgG1 and the light chain constant regions from human kappa chains; 2) h011-3133-YTE: h011-3133-IgG1-YTE format, i.e., humanized sequence combination h011-3133, in combination with the heavy chain constant regions from mutant human IgG1 (YTE mutant) and the light chain constant regions from human kappa chains. The mutant human IgG1 can also be another format of mutation.

The mutated antibody was tested for its affinity with BIAcore (test 6). The results are shown in Table 16.

h011-3133-IgG1

The amino acid sequence of heavy chain: IgG1
SEQ ID NO: 71
QVQLVQSGAEVKKPGASVKVSCKASGYTFTGYEIHWVRQAPGQGLEWMG
YINPSATYTKFNQKFKDRVTMTRDTSISTAYMELSRLRSDDTAVYYCAR
ERIYSNYWFFDVWGQGTTVTVSSASTKGPSVFPLAPSSKSTSGGTAALG
CLVKDYFPEPVTVSWNSGALTSGVHTFPAVLQSSGLYSLSSVVTVPSSS
LGTQTYICNVNHKPSNTKVDKKVEPKSCDKTHTCPPCPAPELLGGPSVF
LFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKA
KGQPREPQVYTLPPSRDELTKNQVSLTCLVKGFYPSDIAVEWESNGQPE
NNYKTTPPVLDSDGSFFLYSKLTVDKSRWQQGNVFSCSVMHEALHNHYT
QKSLSLSPGK
Heavy chain DNA sequence:
SEQ ID NO: 72
ATGGAGTTTGGGCTGAGCTGGCTTTTTCTTGTCGCGATTCTTAAGGGTG
TCCAGTGCCAGGTGCAGCTGGTGCAGTCTGGCGCCGAGGTGAAGAAGCC
CGGAGCATCTGTGAAGGTGTCTTGTAAGGCCTCTGGCTATACCTTTACC
GGCTACGAGATCCACTGGGTGCGGCAGGCACCCGGGCAGGGCCTGGAGT
GGATGGGCTACATCAACCCCTCTGCTACCTACACCAAGTTTAACCAGAA
GTTCAAGGACCGGGTGACCATGACCCGGGACACCTCTATCTCTACCGCC
TACATGGAGCTGTCTCGGCTGCGGTCTGACGACACCGCCGTGTACTACT
GCGCACGCGAACGGATCTACTCTAACTACTGGTTCTTCGACGTGTGGGG
CCAGGGCACCACCGTGACCGTGTCTTCTGCTTCGACCAAGGGCCCATCG
GTCTTCCCCCTGGCACCCTCCTCCAAGAGCACCTCTGGGGGCACAGCGG
CCCTGGGCTGCCTGGTCAAGGACTACTTCCCCGAACCGGTGACGGTGTC
GTGGAACTCAGGCGCCCTGACCAGCGGCGTGCACACCTTCCCGGCTGTC
CTACAGTCCTCAGGACTCTACTCCCTCAGCAGCGTGGTGACCGTGCCCT
CCAGCAGCTTGGGCACCCAGACCTACATCTGCAACGTGAATCACAAGCC
CAGCAACACCAAGGTGGACAAGAAAGTTGAGCCCAAATCTTGTGACAAA
ACTCACACATGCCCACCGTGCCCAGCACCTGAACTCCTGGGGGGACCGT
CAGTCTTCCTCTTCCCCCCAAAACCCAAGGACACCCTCATGATCTCCCG
GACCCCTGAGGTCACATGCGTGGTGGTGGACGTGAGCCACGAAGACCCT
GAGGTCAAGTTCAACTGGTACGTGGACGGCGTGGAGGTGCATAATGCCA
AGACAAAGCCGCGGGAGGAGCAGTACAACAGCACGTACCGTGTGGTCAG
CGTCCTCACCGTCCTGCACCAGGACTGGCTGAATGGCAAGGAGTACAAG
TGCAAGGTCTCCAACAAAGCCCTCCCAGCCCCCATCGAGAAAACCATCT
CCAAAGCCAAAGGGCAGCCCCGAGAACCACAGGTGTACACCCTGCCCCC
ATCCCGGGATGAGCTGACCAAGAACCAGGTCAGCCTGACCTGCCTGGTC
AAAGGCTTCTATCCCAGCGACATCGCCGTGGAGTGGGAGAGCAATGGGC
AGCCGGAGAACAACTACAAGACCACGCCTCCCGTGCTGGACTCCGACGG
CTCCTTCTTCCTCTACAGCAAGCTCACCGTGGACAAGAGCAGGTGGCAG
CAGGGGAACGTCTTCTCATGCTCCGTGATGCATGAGGCTCTGCACAACC
ACTACACGCAGAAGAGCCTCTCCCTGTCTCCGGGTAAATGA
h011-3133-kappa
The amino acid sequence of light chain: kappa
light chain
SEQ ID NO: 73
DIVMTQSPSSLSASVGDRVTITCKASQNVYTAVAWYQQKPGKSPKLLIY
SASNRYTGVPSRFSGSGSGTDFTLTISSLQPEDFATYFCQQYSSYPYTF
GGGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC
Light chain DNA sequence:
SEQ ID NO: 74
ATGGACATGCGCGTGCCCGCCCAGCTGCTGGGCCTGCTGCTGCTGTGGT
TCCCCGGCTCGCGATGCGACATCGTGATGACCCAGTCTCCCTCATCTCT
GAGTGCCTCTGTTGGCGACCGGGTGACCATCACCTGCAAAGCCTCTCAG
AACGTATACACAGCCGTGGCCTGGTATCAACAGAAGCCCGGCAAGTCCC
CCAAGCTGCTGATTTACTCTGCCTCTAACCGGTACACCGGCGTGCCCTC
TCGGTTCTCTGGCTCTGGTTCTGGCACCGACTTCACCCTGACTATCTCT
TCTCTGCAGCCCGAGGACTTCGCCACCTACTTCTGCCAGCAGTACTCTT
CTTACCCCTACACCTTCGGCGGAGGCACCAAGGTGGAGATCAAGCGTAC
GGTGGCTGCACCATCTGTCTTCATCTTCCCGCCATCTGATGAGCAGTTG
AAATCTGGAACTGCCTCTGTTGTGTGCCTGCTGAATAACTTCTATCCCA
GAGAGGCCAAAGTACAGTGGAAGGTGGATAACGCCCTCCAATCGGGTAA
CTCCCAGGAGAGTGTCACAGAGCAGGACAGCAAGGACAGCACCTACAGC
CTCAGCAGCACCCTGACGCTGAGCAAAGCAGACTACGAGAAACACAAAG
TCTACGCCTGCGAAGTCACCCATCAGGGCCTGAGCTCGCCCGTCACAAA
GAGCTTCAACAGGGGAGAGTGTTGA
h011-3133-IgGl-YTE
(light chain: h011-3133-kappa SEQ ID NO: 73)
The amino acid sequence of heavy chain:
IgG1-YTE
SEQ ID NO: 75
QVQLVQSGAEVKKPGASVKVSCKASGYTFTGYEIHWVRQAPGQGLEWMG
YINPSATYTKFNQKFKDRVTMTRDTSISTAYMELSRLRSDDTAVYYCAR
ERIYSNYWFFDVWGQGTTVTVSSASTKGPSVFPLAPSSKSTSGGTAALG
CLVKDYFPEPVTVSWNSGALTSGVHTFPAVLQSSGLYSLSSVVTVPSSS
LGTQTYICNVNHKPSNTKVDKKVEPKSCDKTHTCPPCPAPELLGGPSVF
LFPPKPKDTLYITREPEVTCVVVDVSHEDPEVKFNWYVDGVEVHNAKTK
PREEQYNSTYRVVSVLTVLHQDWLNGKEYKCKVSNKALPAPIEKTISKA
KGQPREPQVYTLPPSRDELTKNQVSLTCLVKGFYPSDIAVEWESNGQPE
NNYKTTPPVLDSDGSFFLYSKLTVDKSRWQQGNVFSCSVMHEALHNHYT
QKSLSLSPGK
Heavy chain DNA sequence:
SEQ ID NO:76
ATGGAGTTTGGGCTGAGCTGGCTTTTTCTTGTCGCGATTCTTAAGGGTG
TCCAGTGCCAGGTGCAGCTGGTGCAGTCTGGCGCCGAGGTGAAGAAGCC
CGGAGCATCTGTGAAGGTGTCTTGTAAGGCCTCTGGCTATACCTTTACC
GGCTACGAGATCCACTGGGTGCGGCAGGCACCCGGGCAGGGCCTGGAGT
GGATGGGCTACATCAACCCCTCTGCTACCTACACCAAGTTTAACCAGAA
GTTCAAGGACCGGGTGACCATGACCCGGGACACCTCTATCTCTACCGCC
TACATGGAGCTGTCTCGGCTGCGGTCTGACGACACCGCCGTGTACTACT
GCGCACGCGAACGGATCTACTCTAACTACTGGTTCTTCGACGTGTGGGG
CCAGGGCACCACCGTGACCGTGTCTTCTGCTTCGACCAAGGGCCCATCG
GTCTTCCCCCTGGCACCCTCCTCCAAGAGCACCTCTGGGGGCACAGCGG
CCCTGGGCTGCCTGGTCAAGGACTACTTCCCCGAACCGGTGACGGTGTC
GTGGAACTCAGGCGCCCTGACCAGCGGCGTGCACACCTTCCCGGCTGTC
CTACAGTCCTCAGGACTCTACTCCCTCAGCAGCGTGGTGACCGTGCCCT
CCAGCAGCTTGGGCACCCAGACCTACATCTGCAACGTGAATCACAAGCC
CAGCAACACCAAGGTGGACAAGAAAGTTGAGCCCAAATCTTGTGACAAA
ACTCACACATGCCCACCGTGCCCAGCACCTGAACTCCTGGGGGGACCGT
CAGTCTTCCTCTTCCCCCCAAAACCCAAGGACACCCTCTACATCACCCG
GGAGCCTGAGGTCACATGCGTGGTGGTGGACGTGAGCCACGAAGACCCT
GAGGTCAAGTTCAACTGGTACGTGGACGGCGTGGAGGTGCATAATGCCA
AGACAAAGCCGCGGGAGGAGCAGTACAACAGCACGTACCGTGTGGTCAG
CGTCCTCACCGTCCTGCACCAGGACTGGCTGAATGGCAAGGAGTACAAG
TGCAAGGTCTCCAACAAAGCCCTCCCAGCCCCCATCGAGAAAACCATCT
CCAAAGCCAAAGGGCAGCCCCGAGAACCACAGGTGTACACCCTGCCCCC
ATCCCGGGATGAGCTGACCAAGAACCAGGTCAGCCTGACCTGCCTGGTC
AAAGGCTTCTATCCCAGCGACATCGCCGTGGAGTGGGAGAGCAATGGGC
AGCCGGAGAACAACTACAAGACCACGCCTCCCGTGCTGGACTCCGACGG
CTCCTTCTTCCTCTACAGCAAGCTCACCGTGGACAAGAGCAGGTGGCAG
CAGGGGAACGTCTTCTCATGCTCCGTGATGCATGAGGCTCTGCACAACC
ACTACACGCAGAAGAGCCTCTCCCTGTCTCCGGGTAAATGA

### Test 1 ELISA Experiment for PCSK9 Antibody Binding to PCSK9

The binding ability of anti-PCSK9 antibodies of the present invention to PCSK9 protein (WT PCSK9, SEQ ID NO: 5) was detected by measuring the amounts of antibodies binding to PCSK9 immobilized on the ELISA plate.

Streptavidin (sigma, CAT #S4762) was diluted to 2 μg/ml with PBS and was added into a 96-well ELISA plate, at 4° C. for overnight. The plate was washed and then was blocked with Tris buffer (including 0.9 mM CaCl₂, 0.05% Tween 20 and 5% skim milk) at 37° C. for 2 hours. Then the plate was washed again and 100 μl/well of biotin-labeled PCSK9 (produced in-house, diluted with Tris buffer containing 0.9 mM CaCl₂, 0.05% Tween 20 and 1% skim milk) was added and incubated at 37° C. for 1 hour. After the wash step various concentrations of diluted PCSK9 antibody samples was added to the plate and incubated at 37° C. for 1 hour. Then the plate was washed again and HRP-goat-anti-human (H+L) antibody (Jackson ImmunoResearch, CAT #109-035-088) was added and incubated at 37° C. for 1 hour. Then the plate was washed and tetramethyl brenzidine solution was added for development. Finally, the stop buffer was added and the OD450 value was measured on the Microplate reader, and then EC50 was calculated.

The results of ELISA test for detecting the binding ability of humanized and affinity maturation PCSK9 antibodies according to the present invention to human PCSK9 are shown in table 12.

TABLE 12
Binding Assay of PCSK9 antibodies according
to the present invention to PCSK9

| Clone No. | EC50 (μg/ml) |
|---|---|
| h011-3050 | 0.00312 |
| h011-3058 | 0.00369 |
| h011-3065 | 0.00539 |
| h011-3070 | 0.00511 |
| h011-3073 | 0.00389 |
| h011-3093 | 0.00381 |
| h011-3095 | 0.00441 |
| h011-3111 | 0.00498 |
| h011-3118 | 0.00351 |
| h011-3120 | 0.00538 |
| h011-3121 | 0.00378 |
| h011-3133 | 0.00435 |
| h011-3147 | 0.00483 |
| h011-3174 | 0.00529 |
| h011-3181 | 0.00221 |
| h011-3187 | 0.00450 |
| h011-3190 | 0.00500 |
| h011-3191 | 0.00631 |
| h011-3192 | 0.00664 |
| h011-3193 | 0.00470 |
| h011-3194 | 0.00469 |
| h011-3195 | 0.00679 |
| h011-3 | 0.00719 |

The data showed that the humanized antibodies according to the present invention have excellent binding activity to PCSK9.

### Test 2 ELISA for Binding of PCSK9 Antibodies to PCSK9-Y

The binding ability of anti-PCSK9 antibodies of the present invention to PCSK9-Y protein (mutant PCSK9, SEQ ID NO: 6) was detected by measuring the amounts of antibodies binding to PCSK9-Y immobilized on the ELISA plate.

Streptavidin (sigma, CAT #S4762) was diluted to 2 μg/ml with PBS and was added into a 96-well ELISA plate, at 4° C. for overnight. The plate was washed and then was blocked with Tris buffer (including 0.9 mM CaCl₂, 0.05% Tween 20 and 5% skim milk) at 37° C. for 2 hours. The plate was washed and 100 μl/well of biotin-labeled PCSK9-Y (produced in-house, diluted with Tris buffer containing 0.9 mM CaCl₂, 0.05% Tween 20 and 1% skim milk) was added and incubated at 37° C. for 1 hour. After the wash step, various concentrations of diluted PCSK9 antibody samples were added to the plate and incubated at 37° C. for 1 hour. Then the plate was washed again and HRP-goat-anti-human (H+L) antibody (Jackson ImmunoResearch, CAT #109-035-088) was added and incubated at 37° C. for 1 hour. Then the plate was washed and tetramethyl brenzidine solution was added for development. Finally, the stop buffer was added and the OD450 value was measured on the Microplate reader, and then EC50 was calculated.

The results of ELISA test for detecting the binding ability of humanized and affinity maturation PCSK9 antibodies according to the present invention to human PCSK9-Y are shown in table 13.

TABLE 13
Binding Assay of PCSK9 antibodies according
to the present invention to PCSK9-Y

| Clone No | EC50 (μg/ml) |
|---|---|
| h011-3050 | 0.00525 |
| h011-3058 | 0.00606 |
| h011-3065 | 0.01092 |
| h011-3070 | 0.01444 |
| h011-3073 | 0.01227 |
| h011-3093 | 0.00989 |
| h011-3095 | 0.01323 |
| h011-3111 | 0.00681 |
| h011-3118 | 0.00851 |
| h011-3120 | 0.00711 |
| h011-3121 | 0.00695 |
| h011-3133 | 0.00492 |
| h011-3147 | 0.00668 |
| h011-3174 | 0.01123 |
| h011-3181 | 0.00678 |
| h011-3187 | 0.00704 |
| h011-3190 | 0.00478 |
| h011-3191 | 0.00660 |
| h011-3192 | 0.12670 |
| h011-3193 | 0.02210 |
| h011-3194 | 0.01685 |
| h011-3195 | 0.02178 |
| h011-3 | 1.77950 |

The data showed that the humanized antibodies according to the present invention have excellent binding activity to PCSK9-Y.

### Test 3 Anti-PCSK9 Antibodies Block the Binding of LDLR-FC/PCSK9-Y

The blocking ability of PCSK9 antibodies according to the present invention for the binding of LDLR-FC and PCSK9-Y (mutant PCSK9, SEQ ID NO: 6) was tested by measuring the amount of PCSK9-Y binding to LDLR in the presence of the antibodies.

LDLR-FC (produced in-house, with sequence of SEQ ID NO: 8) was diluted to 2 μg/ml with phosphate buffer and was added into the 96-well ELISA plate, at 4° C. overnight. The plate was washed and then blocked with Tris buffer (including 0.9 mM CaCl₂, 0.05% Tween 20 and 5% skim milk) at 37° C. for 2 hours. Then the plate was washed and 100 μl/well of mixture of biotin-labeled mutant PCSK9 (diluted to 1 μg/ml with Tris buffer containing 0.9 mM CaCl₂, 0.05% Tween 20 and 1% skim milk) and antibody samples (diluted with Tris buffer containing 0.9 mM CaCl₂, 0.05% Tween 20 and 1% skim milk) was added and incubated at 37° C. for 1 hour. Then the plate was washed again and horseradish peroxidase-streptavidin (sigma, CAT #S2438) was added and incubated at 37° C. for 1 hour. Then the plate was washed and tetramethyl brenzidine solution was added for development. Finally, the stop buffer was added and OD450 value was measured on the Microplate reader, then IC50 value was calculated.

The results of blocking test of the humanized and affinity maturation antibodies according to the present invention to the binding of LDLR-FC/PCSK9-Y, are shown in table 14.

TABLE 14
Blocking test of PCSK9 antibodies
to the binding of PCSK9-Y and LDLR

| Clone No. | IC50 (μg/ml) |
|---|---|
| h011-3050 | 0.123 |
| h011-3058 | 0.162 |
| h011-3065 | 0.312 |
| h011-3070 | 0.228 |
| h011-3073 | 0.129 |
| h011-3093 | 0.287 |
| h011-3095 | 0.391 |
| h011-3111 | 0.226 |
| h011-3118 | 0.230 |
| h011-3120 | 0.213 |
| h011-3121 | 0.174 |
| h011-3133 | 0.175 |
| h011-3147 | 0.173 |
| h011-3174 | 0.230 |
| h011-3181 | 0.233 |
| h011-3187 | 0.156 |
| h011-3190 | 0.222 |
| h011-3191 | 0.226 |
| h011-3192 | 2.360 |
| h011-3193 | 0.871 |
| h011-3194 | 0.579 |
| h011-3195 | 0.689 |
| h011-3 | 5.489 |

The data showed that the PCSK9 antibodies according to the present invention can effectively block the binding of PCSK9-Y to LDLR.

The method above was also used to test the blocking effect of PCSK9 antibodies according to the present invention to the binding of other formats of LDLR-FC (produced in-house, with sequence of SEQ ID NO: 7 or SEQ ID NO: 9) to PCSK9 (SEQ ID NO: 5). The results showed that the PCSK9 antibodies according to the present invention can effectively block the binding of PCSK9 to truncated LDLR.

### Test 4 Anti-PCSK9 Antibodies Block the Binding of LDLR-FC/PCSK9

The blocking ability of PCSK9 antibodies according to the present invention for the binding of LDLR-FC (produced in-house, with sequence of SEQ ID NO: 8) and PCSK9 (SEQ ID NO: 5) was tested by measuring the amount of PCSK9 binding to LDLR in the presence of the antibodies.

LDLR-FC was diluted to 5 μg/ml with phosphate buffer and was added into the 96-well ELISA plate, kept at 4° C. overnight. The plate was washed and then blocked with Tris buffer (including 0.9 mM CaCl₂, 0.05% Tween 20 and 5% skim milk) at 37° C. for 2 hours. Then the plate was washed and 100 μl/well of mixture of biotin-labeled PCSK9 (diluted to 2 μg/ml with Tris buffer containing 0.9 mM CaCl₂, 0.05% Tween 20 and 1% skim milk) and antibody samples (diluted with Tris buffer containing 0.9 mM CaCl₂, 0.05% Tween 20 and 1% skim milk) was added and incubated at 37° C. for 1 hour. Then the plate was washed again and horseradish peroxidase-streptavidin (sigma, CAT #S2438) was added and incubated at 37° C. for 1 hour. Then the plate was washed and tetramethyl brenzidine solution was added for development. Finally, the stop buffer was added and OD450 value was measured on the Microplate reader, then IC50 value was calculated.

The results of blocking test of the humanized and affinity maturation antibodies according to the present invention to the binding of LDLR-FC/PCSK9, are shown in table 15.

TABLE 15
Blocking test of PCSK9 antibodies
to the binding of PCSK9 to LDLR

| Clone No. | IC50 (μg/ml) |
|---|---|
| h011-3050 | 0.598 |
| h011-3058 | 0.542 |
| h011-3065 | 0.730 |
| h011-3070 | 0.629 |
| h011-3073 | 0.604 |
| h011-3093 | 0.706 |
| h011-3095 | 0.582 |
| h011-3111 | 1.224 |
| h011-3118 | 1.042 |
| h011-3120 | 0.911 |
| h011-3121 | 0.662 |
| h011-3133 | 0.495 |
| h011-3147 | 0.567 |
| h011-3174 | 1.671 |
| h011-3181 | 0.857 |
| h011-3187 | 0.666 |
| h011-3190 | 0.837 |
| h011-3191 | 0.740 |
| h011-3192 | 0.698 |
| h011-3193 | 0.621 |
| h011-3194 | 0.628 |
| h011-3195 | 2.252 |
| h011-3 | 0.681 |

The data showed that the PCSK9 antibodies according to the present invention can effectively block the binding of PCSK9 to LDLR.

The method above was also used to test the blocking effect of PCSK9 antibodies according to the present invention on the binding of other formats of LDLR-FC (produced in-house, with sequence of SEQ ID NO: 7 or SEQ ID NO: 9) to PCSK9 (SEQ ID NO: 5). The results showed that the PCSK9 antibodies according to the present invention can effectively block the binding of PCSK9 to truncated LDLR formats.

### Test 5 LDL Uptake of PCSK9 Antibodies

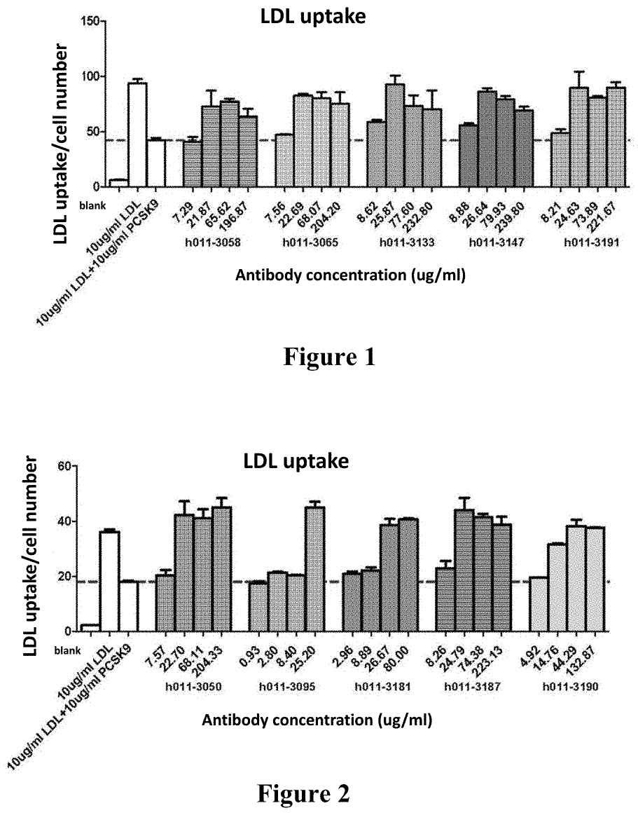

HepG2 cells (Chinese Academy of Sciences cell bank, # CAT , TCHu72) were cultured in DMEM medium (Hyclone, # CAT SH30243.01B) (containing 10% FBS, Gibco, # CAT 10099-141). When the cells covered 80-90% of the plate, the cells were digested, suspended and counted, 1.5*10⁴ cells/well were plated in a 96-well plate. 24 h later, the medium was replaced with DMEM and 10% serum without lipoprotein (Millipore, CAT #LP4). 48 h later, the plate was washed twice with PBS buffer, then a mixture, which was pre-incubated at 4° C. for 1 hour, of PCSK9 (SEQ ID NO: 1, at a final concentration of 10 μg/ml), antibody samples (diluted to various concentrations with the medium), and BODIPY-® LDL at a final concentration of 10 μg/ml (Invitrogen, CAT #L3483) was added to the plate. After being incubated at 37° C. for 6 hours, the plate was washed twice with PBS buffer. The fluorescence value was determined on a Microplate reader (EX485 nm/ EM535 nm), then 50 μl/well of CellTiter-Glo® Cell Activity Luminescence Detection Reagent (Promega, G7571) was added, and the chemiluminescence value was determined. LDL uptake results are shown in FIGS. 1 and 2, which indicated that PCSK9 antibodies according to the present invention can promote the LDL uptake by HepG2 cells.

### Test 6 BIAcore Assay for PCSK9 Antibody Affinity

According to the method described in the human Fab capture kit (Cat. #28-9583-25, GE), the human Fab capture molecule was covalently linked to the CM5 biochip (Cat. #BR-1000-12, GE) so that the PCSK9 antibodies of the present invention were affinity captured. Then, human PCSK9 antigen (PCSK9 with His tag: PCSK9-His6, SEQ ID NO: 1) flowed through the surface of the biochip, and the reaction signal was detected in real time using a Biacore instrument to obtain the association and dissociation curves. Finally, the affinity values were obtained by fitting and are shown at table 16 below. After each cycle of dissociation was finished, the biochip was washed and regenerated with regeneration solution in the human Fab capture kit (GE).

TABLE 16
Affinity of PCSK9 Monoclonal Antibodies

| Stationary phase | Mobile phase | Affinity(M) |
|---|---|---|
| h011-3133-WT | huPCSK9 | <5.26E−11 |
| h011-3133-YTE | | <5.18E−11 |

The result demonstrated that the PCSK9 antibodies of present invention have strong affinity to human PCSK9 antigen.

The same method was used to detect the affinity of PCSK9 antibodies to PCSK9-Y (SEQ ID NO: 4), and the results demonstrated that the PCSK9 antibodies of present invention have strong affinity to PCSK9-Y antigen.

### Test 7 Pharmacodynamic Test: PCSK9 Antibody Reduced the LDL-c In Vivo

A PCSK9-overexpressing mouse model was built up and the mice were injected with PCSK9 antibody via tail vein. The effect of the PCSK9 antibodies according to the present invention on reducing LDL-c level in vivo in PCSK9-overexpressing mice was evaluated. Human IgG (human immunoglobulin purified from the mixed normal human serum by traditional affinity chromatography, such as Protein A) was used as a blank control.

C57Bl/6 mice (purchased from Shanghai Sippr-BK Laboratory Animal Co., Ltd.) were adapted for 5 days in the laboratory environment, and injected with 4×10¹¹ v.g. of AAV-PCSK9 virus (Benyuan Zhengyang Gene Technology Co., Ltd.) via tail vein. 10 days after the virus injection, the mice were fasted overnight. Then blood was taken from the eyelid and LDL-c was measured by HDL and LDL/VLDL Cholesterol Quantification Kit (purchased from BioVision, catalog number #K613-100). Mice were randomly divided into groups (6 mice/group (n=6)) according to the concentration of LDL-c and were administered with antibodies via tail vein injection. Human IgG and PCSK9 antibody, produced in-house, were administered at a dose of 10 mg/kg (human IgG and PCSK9 antibody were both prepared in PBS at a concentration of 1 mg/ml). The mice were fasted overnight before blood sampling. 24 h, 48 h, and 72 h after administration, blood was taken from the eyelids, kept at 37° C. for 1 hour, centrifuged at 3500 rpm for 10 minutes, and the serum was stored at −80° C.

After the last serum collection, all the frozen serum were tested on the same day. The concentration of LDL-c in the serum were measured by HDL and LDL/VLDL Cholesterol Quantification Kit in accordance with kit instructions.

### Discussion:

1. Pharmacodynamic Test: PCSK9 antibodies of Group h011-3133 reduced LDL-c level in vivo

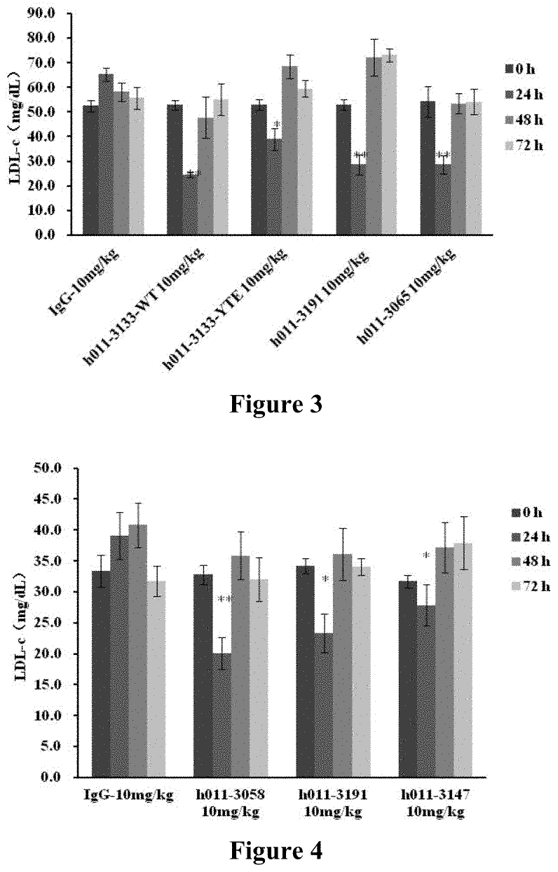

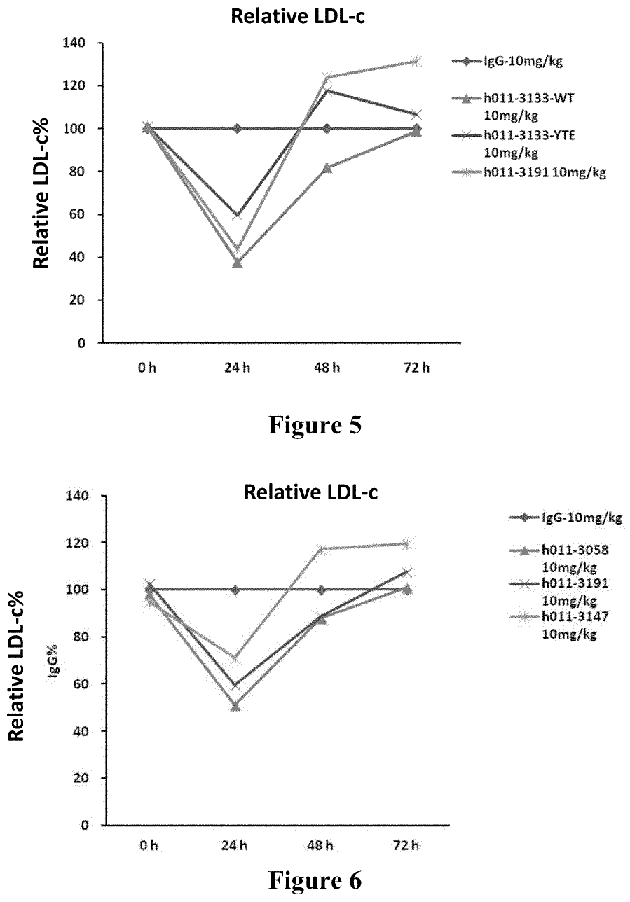

10 days after the injection of the AAV8-PCSK9 virus, the concentrations of LDL-c in the serum were averaged as 53 mg/dl. 24 h after administration of antibodies to the divided groups, the concentrations of LDL-c of PCSK9 antibody groups of h011-3133-WT, h011-3133-YTE, h011-3191, h011-3065 were decreased by 62%, 40%, 56%, and 56%, respectively, compared to that of the IgG group. Each antibody-dosing group significantly reduced the concentration of LDL-c in the serum at a dose of 10 mg/kg and there was no significant difference between the antibody-dosing groups. 48 h after administration of antibodies, the concentration of LDL-c in the h011-3133-WT group was decreased by 18% compared to the IgG group, and there was no significant difference from the IgG group. The other groups were comparable to the IgG group. 72 h after administration of antibodies, the concentration of LDL-c in each group was comparable to that of the IgG group. The results are shown in FIG. 3, Table 17 and FIG. 5. FIG. 3 shows the absolute value of serum LDL-c at different time points after administration of each sample. FIG. 5 shows the percentage of LDL-c serum content of antibody-dosing group relative to IgG group at the same time point, and the value in IgG group was used as control of 100%.

In FIG. 4, the concentration of LDL-c in the serum of normal mouse is about 6 mg/dl. 34 days after the injection of AAV8-PCSK9 virus, the concentrations of LDL-c in the serum were averaged as 33 mg/dl. Mice were divided into groups and antibodies were administered immediately, 24 h after administration, the concentrations of LDL-c in h011-3058, h011-3191, h011-3147 groups were decreased by 49%, 40% and 29%, respectively, compared to the IgG group. h011-3058, h011-3191, h011-3147 significantly reduced the concentration of LDL-c in the serum at a dose of 10 mg/kg and there was no significant difference between the antibody-dosing groups. 48 h after administration, the concentrations of LDL-c in the h011-3058, h011-3191, h011-3147 groups were decreased by 12%, 11% and 9%, respectively, compared to the IgG group, and there was no significant difference from the IgG group. 72 h after administration, the concentration of LDL-c in each group was comparable to that of the IgG group. The results are shown in FIG. 4, Table 18 and FIG. 6. FIG. 4 shows the absolute value of serum LDL-c at different time points after administration of each sample. FIG. 6 shows the percentage of LDL-c serum content of antibody-dosing group relative to IgG group at the same time point, and the value in IgG group was used as control of 100%.

In summary, h011-3133-WT, h011-3133-YTE, h011-3191, h011-3065, h011-3058 and h011-3147 all were able to reduce the concentration of LDL-c in the serum of human PCSK9 overexpressed mice in this experiment.

TABLE 17
Changes in the concentrations of LDL-c in the serum of mice

| LDL-c (mg/dl) | | | | | % IgG | | | |
|---|---|---|---|---|---|---|---|---|
| Unit (10 mg/kg) | 0 h | 24 h | 48 h | 72 h | 0 h | 24 h | 48 h | 72 h |
| IgG | 52.4 ± 2.47 | 65.4 ± 2.66 | 58.3 ± 3.71 | 55.7 ± 4.51 | 100 | 100 | 100 | 100 |
| h011-3133-WT | 52.8 ± 2.12 | 24.6 ± 1.09 | 47.7 ± 8.38 | 55.0 ± 6.35 | 101 | 38 | 82 | 99 |
| h011-3133-YTE | 52.9 ± 2.08 | 39.0 ± 4.40 | 68.5 ± 4.85 | 59.4 ± 3.36 | 101 | 60 | 118 | 107 |
| h011-3191 | 52.9 ± 2.15 | 28.7 ± 4.04 | 72.2 ± 7.39 | 73.2 ± 2.66 | 101 | 44 | 124 | 131 |
| h011-3065 | 54.3 ± 6.30 | 28.6 ± 3.78 | 53.4 ± 4.09 | 54.1 ± 5.21 | 104 | 44 | 92 | 97 |

TABLE 18
Changes in the concentration of LDL-c in the serum of mice

| LDL-c (mg/dl) | | | | | % IgG | | | |
|---|---|---|---|---|---|---|---|---|
| Unit (10 mg/kg) | 0 h | 24 h | 48 h | 72 h | 0 h | 24 h | 48 h | 72 h |
| IgG | 33.3 ± 2.57 | 39.1 ± 3.81 | 40.8 ± 3.56 | 31.7 ± 2.47 | 100 | 100 | 100 | 100 |
| h011-3058 | 32.8 ± 1.59 | 20.0 ± 2.59 | 35.9 ± 3.83 | 32.0 ± 3.49 | 98 | 51 | 88 | 101 |
| h011-3191 | 34.2 ± 1.18 | 23.3 ± 3.13 | 36.1 ± 4.19 | 34.1 ± 1.36 | 103 | 60 | 89 | 108 |
| h011-3147 | 31.7 ± 1.03 | 27.8 ± 3.32 | 37.2 ± 4.13 | 37.9 ± 4.31 | 95 | 71 | 91 | 119 |

### Test 8 Competitive Experiment

In the competitive ELISA experiment, the plate was coated with one antibody overnight. Then biotin-PCSK9-his and a competitive antibody at a concentration 50 times higher than the coating antibody were added. The coating antibody will compete with the competitive antibody to bind to an antigen. The antigen signal at the plate was then tested. The results show that, h011-3133-YTE and 21B12 (U.S. Pat. No. 8,030,457B2) can compete to bind to antigen, however, there is no clear competition binding between the two antibodies, suggesting antigen epitopes of the two antibodies are different.

| IR (%) | h011-3133-YTE | 21B12 |
|---|---|---|
| h011-3133-YTE | 96.17 | −0.28 |
| 21B12 | 3.01 | 97.78 |

Although the present invention has been described in detail with the aid of the drawings and examples for the sake of clarity, the description and examples should not be construed as limiting the scope of the invention. The disclosures of all patents and scientific literature cited herein are hereby incorporated by reference in their entirety.

1. A PCSK9 antibody specifically binding to PCSK9 or an antigen-binding fragment thereof, comprising:
i) a HCDR1 having the sequence of GYX¹IH (SEQ ID NO: 43), wherein X¹ is T, D or E;
ii) a HCDR2 having the sequence of X²IX³PSX⁴TYTKFNQKFKD (SEQ ID NO: 44), wherein X² is Y or E; X³ is N, L, I or V; and X⁴ is S, G or A;
iii) a HCDR3 having the sequence of AREX⁵IX⁶X⁷NYWFFDX⁸ (SEQ ID NO: 45), wherein X⁵ is R or N; X⁶ is Y or F; X⁷ is S or F; and X⁸ is V or R;
iv) a LCDR1 having the sequence of KASQNVYX₁X₂VX₃ (SEQ ID NO: 46), wherein X₁ is T or W; X₂ is A or E; and X₃ is A, D or V;
v) a LCDR2 having the sequence of X₄X₅X₆NRYT (SEQ ID NO: 47), wherein X₄ is S, E or Q; X₅ is A or M; and X₆ is S or V; and
vi) a LCDR3 having the sequence of QQX₇SX₈X₉PX₁₀T (SEQ ID NO: 48), wherein X₇ is Y, F or L; X₈ is S or W; X₉ is Y, F, Q or S; and X₁₀ is Y, D or E.

2.-4. (canceled)

5. The PCSK9 antibody specifically binding to PCSK9 or the antigen-binding fragment thereof according to claim 1, wherein the HCDR1 has the amino acid sequence of SEQ ID NO:14, SEQ ID NO:20, or SEQ ID NO:21, or an amino acid sequence having at least 95% identity to the above sequences;
the HCDR2 has the amino acid sequence of SEQ ID NO: 15, SEQ ID NO: 22, SEQ ID NO: 23, SEQ ID NO: 24, SEQ ID NO: 25, SEQ ID NO: 26, or SEQ ID NO: 27, or an amino acid sequence having at least 95% identity to the above sequences; and
the HCDR3 has the amino acid sequence of SEQ ID NO:16, SEQ ID NO:28, SEQ ID NO:29, or SEQ ID NO:30, or an amino acid sequence having at least 95% identity to the above sequences.

6. The PCSK9 antibody specifically binding to PCSK9 or the antigen-binding fragment thereof according to claim 1, wherein the LCDR1 has the amino acid sequence SEQ ID NO: 17, SEQ ID NO: 31, SEQ ID NO: 32, SEQ ID NO: 33, or SEQ ID NO: 34, or an amino acid sequence having at least 95% identity to the above sequences;
the LCDR2 has the amino acid sequence of SEQ ID NO: 18, SEQ ID NO: 35, SEQ ID NO: 36, or SEQ ID NO: 37, or an amino acid sequence having at least 95% identity to the above sequences; and
the LCDR3 has the amino acid sequence of SEQ ID NO: 19, SEQ ID NO: 38, SEQ ID NO: 39, SEQ ID NO: 40, SEQ ID NO: 41, or SEQ ID NO: 42, or an amino acid sequence having at least 95% identity to the above sequences.

7. The PCSK9 antibody specifically binding to PCSK9 or the antigen-binding fragment thereof according to claim 1, wherein the HCDR1 has the amino acid sequence of SEQ ID NO:14, SEQ ID NO:20, or SEQ ID NO:21, or an amino acid sequence having at least 95% identity to the above sequences;
the HCDR2 has the amino acid sequence of SEQ ID NO: 15, SEQ ID NO: 22, SEQ ID NO: 23, SEQ ID NO: 24, SEQ ID NO: 25, SEQ ID NO: 26, or SEQ ID NO: 27, or an amino acid sequence having at least 95% identity to the above sequences;
the HCDR3 has the amino acid sequence of SEQ ID NO: 16, SEQ ID NO: 28, SEQ ID NO: 29, or SEQ ID NO: 30, or an amino acid sequence having at least 95% identity to the above sequences; and
the LCDR1 has the amino acid sequence of SEQ ID NO: 17, SEQ ID NO: 31, SEQ ID NO: 32, SEQ ID NO: 33, or SEQ ID NO: 34, or an amino acid sequence having at least 95% identity to the above sequences;
the LCDR2 has the amino acid sequence of SEQ ID NO: 18, SEQ ID NO: 35, SEQ ID NO: 36, or SEQ ID NO: 37, or an amino acid sequence having at least 95% identity to the above sequences; and
the LCDR3 has the amino acid sequence of SEQ ID NO: 19, SEQ ID NO: 38, SEQ ID NO: 39, SEQ ID NO: 40, SEQ ID NO: 41, or SEQ ID NO: 42, or an amino acid sequence having at least 95% identity to the above sequences.

8. The PCSK9 antibody specifically binding to PCSK9 or the antigen-binding fragment thereof according to claim 1, wherein the PCSK9 antibody comprises the HCDR1, the HCDR2, the HCDR3, the LCDR1, the LCDR2 and the LCDR3 having the amino acid sequences of:
1) SEQ ID NO: 14, SEQ ID NO: 15, SEQ ID NO: 16, SEQ ID NO: 17, SEQ ID NO: 18, and SEQ ID NO: 19, respectively;
2) SEQ ID NO: 14, SEQ ID NO: 22, SEQ ID NO: 16, SEQ ID NO: 17, SEQ ID NO: 18, and SEQ ID NO: 19, respectively;
3) SEQ ID NO: 14, SEQ ID NO: 23, SEQ ID NO: 16, SEQ ID NO: 17, SEQ ID NO: 18, and SEQ ID NO: 19, respectively;
4) SEQ ID NO: 20, SEQ ID NO: 15, SEQ ID NO: 16, SEQ ID NO: 17, SEQ ID NO: 18, and SEQ ID NO: 19, respectively;
5) SEQ ID NO: 14, SEQ ID NO: 15, SEQ ID NO: 28, SEQ ID NO: 17, SEQ ID NO: 18, and SEQ ID NO: 19, respectively;
6) SEQ ID NO: 14, SEQ ID NO: 15, SEQ ID NO: 29, SEQ ID NO: 17, SEQ ID NO: 18, and SEQ ID NO: 19, respectively;
7) SEQ ID NO: 14, SEQ ID NO: 15, SEQ ID NO: 16, SEQ ID NO: 17, SEQ ID NO: 35, and SEQ ID NO: 19, respectively;
8) SEQ ID NO: 14, SEQ ID NO: 15, SEQ ID NO: 16, SEQ ID NO: 17, SEQ ID NO: 36, and SEQ ID NO: 19, respectively;
9) SEQ ID NO: 14, SEQ ID NO: 15, SEQ ID NO: 16, SEQ ID NO: 31, SEQ ID NO: 18, and SEQ ID NO: 19, respectively;
10) SEQ ID NO: 14, SEQ ID NO: 15, SEQ ID NO: 16, SEQ ID NO: 32, SEQ ID NO: 18, and SEQ ID NO: 19, respectively;
11) SEQ ID NO: 14, SEQ ID NO: 15, SEQ ID NO: 16, SEQ ID NO: 17, SEQ ID NO: 18, and SEQ ID NO: 38, respectively;
12) SEQ ID NO: 14, SEQ ID NO: 15, SEQ ID NO: 16, SEQ ID NO: 17, SEQ ID NO: 18, and SEQ ID NO: 39, respectively;
13) SEQ ID NO: 21, SEQ ID NO: 24, SEQ ID NO: 16, SEQ ID NO: 17, SEQ ID NO: 18, and SEQ ID NO: 19, respectively;
14) SEQ ID NO: 14, SEQ ID NO: 25, SEQ ID NO: 16, SEQ ID NO: 17, SEQ ID NO: 18, and SEQ ID NO: 19, respectively;
15) SEQ ID NO: 14, SEQ ID NO: 15, SEQ ID NO: 30, SEQ ID NO: 17, SEQ ID NO: 37, and SEQ ID NO: 19, respectively;
16) SEQ ID NO: 14, SEQ ID NO: 15, SEQ ID NO: 16, SEQ ID NO: 17, SEQ ID NO: 18, and SEQ ID NO: 40, respectively;
17) SEQ ID NO: 14, SEQ ID NO: 15, SEQ ID NO: 16, SEQ ID NO: 17, SEQ ID NO: 18, and SEQ ID NO: 41, respectively;
18) SEQ ID NO: 14, SEQ ID NO: 26, SEQ ID NO: 16, SEQ ID NO: 17, SEQ ID NO: 18, and SEQ ID NO: 19, respectively;
19) SEQ ID NO: 14, SEQ ID NO: 27, SEQ ID NO: 16, SEQ ID NO: 17, SEQ ID NO: 18, and SEQ ID NO: 19, respectively;
20) SEQ ID NO: 14, SEQ ID NO: 15, SEQ ID NO: 16, SEQ ID NO: 33, SEQ ID NO: 18, and SEQ ID NO: 19, respectively;
21) SEQ ID NO: 14, SEQ ID NO: 15, SEQ ID NO: 16, SEQ ID NO: 34, SEQ ID NO: 18, and SEQ ID NO: 19, respectively;
22) SEQ ID NO: 14, SEQ ID NO: 15, SEQ ID NO: 16, SEQ ID NO: 17, SEQ ID NO: 18, and SEQ ID NO: 42, respectively; or
23) SEQ ID NO: 14, SEQ ID NO: 15, SEQ ID NO: 30, SEQ ID NO: 17, SEQ ID NO: 18, and SEQ ID NO: 19, respectively.

9. The PCSK9 antibody specifically binding to PCSK9 or the antigen-binding fragment thereof according to claim 1, wherein the PCSK9 antibody or the antigen-binding fragment thereof is a murine antibody or fragment thereof.

10. The PCSK9 antibody specifically binding to PCSK9 or the antigen-binding fragment thereof according to claim 1, wherein the PCSK9 antibody or antigen-binding fragment thereof is a chimeric antibody or fragment thereof.

11. The PCSK9 antibody specifically binding to PCSK9 or the antigen-binding fragment thereof according to claim 1, wherein the PCSK9 antibody or antigen-binding fragment thereof is a humanized antibody or fragment thereof.

12. The PCSK9 antibody specifically binding to PCSK9 or the antigen-binding fragment thereof according to claim 11, wherein the humanized antibody comprises a heavy chain variable FR region, wherein the heavy chain variable FR region is derived from a combination sequence of human germline heavy chains IGHV1-2*02 and hjh6.1 or a mutant sequence thereof; wherein the heavy chain variable FR region comprises a FR1, a FR2, a FR3 region from IGHV1-2*02 and a FR4 region from hjh6.1 or a mutant sequence thereof.

13. The PCSK9 antibody specifically binding to PCSK9 or the antigen-binding fragment thereof according to claim 12, wherein the humanized antibody comprises a heavy chain variable region having the amino acid sequence of SEQ ID NO: 10 or a variant thereof; wherein the variant comprises 1, 2, 3, 4, 5, 6, 7, 8, 9 or 10 amino acid changes in SEQ ID NO: 10.

14. The PCSK9 antibody specifically binding to PCSK9 or antigen-binding fragment thereof according to claim 12, wherein the humanized antibody comprises a heavy chain FR region with 1-10 amino acid back-mutations, wherein the back-mutation is one or more back-mutations selected from the group consisting of R72A, T74K, V68A, M70L, M48V, G49A, R67K and R38K.

15. The PCSK9 antibody specifically binding to PCSK9 or the antigen-binding fragment thereof according to claim 1, wherein the PCSK9 antibody comprises a VH, and the VH has the amino acid sequence of SEQ ID NO: 12, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, or SEQ ID NO: 58, or an amino acid sequence having at least 95% identity to the above sequences.

16. The PCSK9 antibody specifically binding to PCSK9 or the antigen-binding fragment thereof according to claim 11, wherein the humanized antibody comprises a light chain FR region on a light chain variable region, wherein the sequence of the light chain FR region is derived from a combination sequence of human germline light chains IGKV1-39*01 and hjk4.1 or a mutant sequence thereof; wherein the light chain FR region comprises a FR1, a FR2, a FR3 from human germline light chain IGKV1-39*01 and a FR4 from hjk4.1, or the mutant sequence thereof.

17. The PCSK9 antibody specifically binding to PCSK9 or the antigen-binding fragment thereof according to claim 16, wherein the humanized antibody comprises a light chain variable region having the amino acid sequence of SEQ ID NO: 11 or a variant thereof; wherein the variant comprises 1, 2, 3, 4, 5, 6, 7, 8, 9 or 10 amino acid changes in SEQ ID NO: 11.

18. The PCSK9 antibody specifically binding to PCSK9 or the antigen-binding fragment thereof according to claim 16, wherein the humanized antibody comprises a light chain FR region with 1-10 amino acid back-mutations, wherein the back-mutation is one or more back-mutations selected from the group consisting of Q3 V, A43S, and Y87F.

19. The PCSK9 antibody specifically binding to PCSK9 or the antigen-binding fragment thereof according to claim 1, wherein the PCSK9 antibody comprises a VL, and the VL has the amino acid sequence of SEQ ID NO: 13, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, or SEQ ID NO: 70, or an amino acid sequence having at least 95% identity to the above sequences.

20. The PCSK9 antibody specifically binding to PCSK9 or the antigen-binding fragment thereof according to claim 11, wherein the PCSK9 antibody comprises a VH and a VL selected from the group consisting of:
1) the VH of SEQ ID NO: 12 and the VL of SEQ ID NO: 13;
2) the VH of SEQ ID NO: 49 and the VL of SEQ ID NO: 13;
3) the VH of SEQ ID NO: 50 and the VL of SEQ ID NO: 13;
4) the VH of SEQ ID NO: 51 and the VL of SEQ ID NO: 13;
5) the VH of SEQ ID NO: 52 and the VL of SEQ ID NO: 13;
6) the VH of SEQ ID NO: 53 and the VL of SEQ ID NO: 13;
7) the VH of SEQ ID NO: 12 and the VL of SEQ ID NO: 59;
8) the VH of SEQ ID NO: 12 and the VL of SEQ ID NO: 60;
9) the VH of SEQ ID NO: 12 and the VL of SEQ ID NO: 61;
10) the VH of SEQ ID NO: 12 and the VL of SEQ ID NO: 62;
11) the VH of SEQ ID NO: 12 and the VL of SEQ ID NO: 63;
12) the VH of SEQ ID NO: 12 and the VL of SEQ ID NO: 64;
13) the VH of SEQ ID NO: 54 and the VL of SEQ ID NO: 13;
14) the VH of SEQ ID NO: 55 and the VL of SEQ ID NO: 13;
15) the VH of SEQ ID NO: 56 and the VL of SEQ ID NO: 65;
16) the VH of SEQ ID NO: 12 and the VL of SEQ ID NO: 66;
17) the VH of SEQ ID NO: 12 and the VL of SEQ ID NO: 67;
18) the VH of SEQ ID NO: 57 and the VL of SEQ ID NO: 13;
19) the VH of SEQ ID NO: 58 and the VL of SEQ ID NO: 13;
20) the VH of SEQ ID NO: 12 and the VL of SEQ ID NO: 68;
21) the VH of SEQ ID NO: 12 and the VL of SEQ ID NO: 69;
22) the VH of SEQ ID NO: 12 and the VL of SEQ ID NO: 70; and
23) the VH of SEQ ID NO: 56 and the VL of SEQ ID NO: 13.

21. The PCSK9 antibody specifically binding to PCSK9 or the antigen-binding fragment thereof according to claim 11, wherein the PCSK9 antibody or antigen-binding fragment thereof further comprises a heavy chain constant region derived from human IgG1, IgG2, IgG3, or IgG4 or a variant thereof, or an amino acid sequence having at least 95% identity to sequences thereof;
wherein the PCSK9 antibody or antigen-binding fragment thereof further comprises a light chain constant region derived from a human κ chain, human λ chain or a variant thereof, or an amino acid sequence having at least 95% identity to the sequence thereof.

22. The PCSK9 antibody specifically binding to PCSK9 or the antigen-binding fragment thereof according to claim 21, wherein the PCSK9 antibody or antigen-binding fragment thereof comprises:
1) a heavy chain sequence of SEQ ID NO: 71 and a light chain sequence of SEQ ID NO: 73; or
2) a heavy chain sequence of SEQ ID NO: 75 and a light chain sequence of SEQ ID NO: 73.

23. A nucleic acid molecule encoding the PCSK9 antibody or antigen-binding fragment thereof according to claim 1.

24. An expression vector comprising the nucleic acid molecule according to claim 23.

25. A host cell transformed with the expression vector according to claim 24, wherein the host cell is selected from the group consisting of a prokaryotic cell and a eukaryotic cell.

26. A method for preparing a PCSK9 antibody, wherein the method comprises culturing the host cell of claim 25 under conditions suitable for the expression of a nucleic acid molecule encoding the PCSK9 antibody and/or recovering the PCSK9 antibody from the host cell.

27. A pharmaceutical composition comprising a therapeutically effective amount of the PCSK9 antibody or the antigen-binging fragment thereof according to claim 1 and one or more pharmaceutically acceptable carriers, diluents or excipients.

28. A method of treating a PCSK9-mediated disease or condition in a subject in need thereof, wherein the disease or condition is selected from the group consisting of hypercholesterolemia, heart disease, metabolic syndrome, diabetes, coronary heart disease, stroke, cardiovascular disease, Alzheimer's disease, and general dyslipidemia, the method comprising administering to the subject the pharmaceutical composition of claim 27.
